(19) United States
(12) Patent Application Publication
(10) Pub. No.: US 2005/0042681 A1
(43) Pub. Date: Feb. 24, 2005

(54) Methods and formulations for diagnosing, monitoring, staging and treating heart failure

(76) Inventors: Jennifer E. Van Eyk, Baltimore, MD (US); Brian Stanley, Baltimore, MD (US); Irena Neverova, Kingston (CA); Ralf Labugger, Kingston (CA)

Correspondence Address: Licata & Tyrrell P.C., 66 East Main Street, Marlton, NJ, 08053, US

(21) Appl. No.: 10/877,133

(22) Filed: Jun. 25, 2004

Related U.S. Application Data
(60) Provisional application No. 60/554,624, filed on Mar. 19, 2004.
(60) Provisional application No. 60/482,833, filed on Jun. 25, 2003.

(51) Int. Cl. G01N 33/53; C12N 9/12

(52) U.S. Cl. 435/7.1

(57) ABSTRACT

Protein profiles useful in diagnosing, monitoring, staging, evaluating treatments and treating and selecting treatments for heart failure are provided.

This patent application claims the benefit of priority from U.S. Provisional Patent Application No. 60/554,624, filed Mar. 19, 2004 and U.S. Provisional Patent Application No. 60/482,833, filed Jun. 25, 2003, each of which is herein incorporated by reference in its entirety.

### FIELD OF THE INVENTION

The present invention relates to the use of proteins and protein profiles for diagnosing, monitoring, staging, evaluating treatments, and treating heart failure.

### BACKGROUND OF THE INVENTION

Heart failure is a complex disease arising from many triggers, most of which are hemodynamic stressors (e.g. hypertension) and ischemic injuries (e.g. myocardial infarction). The progression of heart failure involves cardiac remodeling, a process comprising time-dependent alterations in ventricular shape, mass and volume (Piano et al. J. Cardiovasc. Nurs. 2000 14:1-23; Molkentin Annu Rev. Physiol. 2000 63:391-426). At the cellular level, cardiac remodeling involves myocyte hypertrophy, proliferation of cells in the extracellular matrix and apoptosis (Piano et al. J. Cardiovasc. Nurs. 2000 14:1-23; Molkentin Annu Rev. Physiol. 2000 63:391-426). Myocyte hypertrophy is characterized by increased expression of genes encoding some contractile proteins, such as P-myosin heavy chain and troponin T (TnT), and non-contractile proteins, such as A-type and B-type natriuretic peptides, increased cell size and cytoskeletal alteration (Piano et al. J. Cardiovasc. Nurs. 2000 14:1-23; Molkentin Annu Rev. Physiol. 2000 63:391-426).

Studies of human and animal models of heart failure suggest depressed myocyte function in the later stages of cardiac failure. The mechanisms that underlie myocyte dysfunction have been suggested to involve alterations in the calcium-handling network, myofilament and cytoskeleton (de Tombe, P. P. Cardiovasc. Res. 1998 37:367-380). For example, in human and animal models of heart failure, sarcoplasmic reticulum calcium-ATPase enzyme activity is reduced, while both mRNA and protein levels of the sarcolemmal Na⁺/Ca²⁺ exchanger are increased. Moreover, there is isoform-switching of TnT, reduced phosphorylation of troponin I (TnI), decreased myofibrillar actomyosin ATPase activity and enhanced microtubule formation in both human and animal models of heart failure.

Initially these changes to the heart are meant to compensate for the diseased parts of the myocardium in order to sustain the body's demand for oxygen and nutrients. However, the compensatory phase of heart failure is limited, and, ultimately, the failing heart is unable to maintain cardiac output adequate to meet the body's needs. Thus, there is a transition from a compensatory phase to a decompensatory phase. In the decompensatory phase, the cascade of changes in the heart continues but is no longer beneficial, moving the patient down the progression of heart failure to a chronic state and eventual death.

There are currently no effective treatments of heart failure. Most treatments are targeted at the later stages of the disease and target the symptoms and not the root cause. The only curative treatment at present is heart transplantation. Unlike other diseases, such as cancer, there are few known therapeutic targets in heart failure, and none for the early stages of the disease. There is a significant need for novel therapeutic targets and novel therapeutic agents for heart failure, particularly in the early stages when treatment to halt the disease progression would provide the greatest benefit to the patient.

From a clinical perspective, the disease is clinically asymptomatic in the compensatory and early decompensatory phases. Outward signs of the disease (such as shortness of breath) do not appear until well into the decompensatory phase. Current diagnosis is based on these outward symptoms. There are no known biochemical markers currently available for the pre-symptomatic diagnosis of the disease. By the time diagnosis occurs, the disease is already well underway. Due to this late diagnosis, 50% of patients die within two years of diagnosis. The 5-year survival rate is less than 30%. There is a significant need for new biochemical markers for the early diagnosis of heart failure.

In the present invention, a proteomic approach to the analysis of a biological sample from a swine model for heart failure and a biological sample from human heart failure patients was used to identify proteins and protein profiles useful in diagnosing, monitoring, staging and treating heart failure and in identifying and monitoring treatments for heart failure.

### SUMMARY OF THE INVENTION

An aspect of the present invention relates to the identification of proteins, altered states of which are indicative of heart failure and various stages of heart failure. Proteins identified herein for the first time to be indicative of heart failure include 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, Membrane-type 1 matrix metalloproteinase cytoplasmic tail-binding protein (MTCBP-1), conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3 and T-complex protein 1 theta subunit. Measurement of altered states of one or more of these proteins provides a unique means for diagnosing, monitoring, and treating heart failure and identifying and monitoring treatments of heart failure.

Additional proteins identified herein, altered states of which are indicative of heart failure include alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.

Another aspect of the present invention relates to unique protein profiles indicative of heart failure and various stages of heart failure. Preferably the profile comprises altered states of one or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3 and T-complex protein 1 theta subunit. Also preferably, the profile comprises altered states of two or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.

Another aspect of the present invention relates to methods for diagnosing heart failure in a subject. In one embodiment, heart failure is diagnosed in the subject by a method comprising detecting an altered state of one or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3 and T-complex protein 1 theta subunit. In another embodiment, heart failure is diagnosed in the subject by a method comprising detecting an altered state of two or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.

Another aspect of the present invention relates to methods for monitoring a subject with heart failure or at risk for heart failure. In one embodiment, this method comprises monitoring a state of one or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3 and T-complex protein 1 theta subunit.

In another embodiment, heart failure is monitored in the subject by a method comprising monitoring states of two or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.

Another aspect of the present invention relates to a method for staging progression of heart failure in a subject suffering from heart failure. In one embodiment, this method comprises detecting in the subject the state of one or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3 and T-complex protein 1 theta subunit, and then comparing the detected state of the protein in the subject with disease controls for the same protein from various stages of heart failure to determine the stage of progression of heart failure of the subject. In another embodiment, heart failure is staged in the subject by a method comprising detecting a state of two or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin. In this embodiment, the detected states of the two or more proteins in the subject are compared to the states of the same two or more proteins from disease controls at various stages of heart failure to determine the stage of progression of heart failure of the subject.

Another aspect of the present invention relates to a method for evaluating treatment of a subject with heart failure. In one embodiment, the method comprises monitoring in the subject changes in the state of one or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3 and T-complex protein 1 theta subunit. In another embodiment, the method comprises monitoring in the subject changes in the state of two or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.

Another aspect of the present invention is to provide a method for treating a subject with heart failure comprising administering to the subject an agent which modulates a state of one or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat-shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.

Another aspect of the present invention is to provide a method for screening for agents potentially useful in treatment of heart failure which comprises assessing the ability of an agent to modulate a state of one or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.

Yet another aspect of the present invention is to provide methods for identifying agents that modulate progression of heart failure. In these methods, protein profiles of the present invention are obtained in the swine model for ischemic heart failure in the presence and absence of a test agent and compared to corresponding proteins profiles for appropriate controls. A change in the protein profile upon administration of the test agent as compared to the control protein profile is indicative of the test agent being a modulator of heart failure.

The inventors expect that the methods of the invention can be carried out by obtaining a profile of one or more substrates and/or metabolites involved in at least one of the glycolytic pathway, the TCA cycle, the citric acid cycle, the beta oxidation pathway, and the electron transport chain. Preferably, the one or more substrates are processed by the above-mentioned enzymes, and the metabolites are metabolites produced by those enzymes.

### DETAILED DESCRIPTION OF THE INVENTION

Heart failure is a progressive condition triggered by an initial insult to the myocardial tissue that results in gradual, yet continual damage to the heart muscle until function ceases. Examples of insults which can trigger heart failure include, but are not limited to, stress, hypoxia, hyperoxia, hypoxemia, infection, trauma, toxins, drugs (e.g. chemotherapeutics with muscle damaging side effects as well as drugs of abuse such as cocaine and alcohol), hypertension, ischemia (inclusive of conditions wherein blood flow to the heart is completely occluded as well as conditions wherein blood flow is decreased as compared to normal flow), ischemia reperfusion, hyperperfusion, hypoperfusion, heart transplantation and/or rejection, surgery, inflammation, and pressure damage such as that caused by atmospheric pressure changes. The term heart failure thus encompasses any condition associated with damage to the myocardium from subtle insult to, for example, myocardial infarction. Insofar as the protein profiles described herein are useful for diagnosing, monitoring, staging, evaluating treatments, and treating heart failure, the profiles may also be useful for diagnosing, monitoring, staging, evaluating treatments, and treating physiological hypertrophy of the heart,.as seen, for example, in althletes' hearts.

After the trigger, a cascade of changes is initiated in the myocardial proteome (the protein content of the heart) of the remaining viable tissue not directly affected by the original trigger. It has now been found that this proteome can be divided into groups of proteins (or subproteomes) that share a common cellular location (e.g., mitochondria) or a common function (e.g., metabolic pathway such as glycolysis), and that the observed changes range from alterations in abundance to post-translational modifications.

The present invention provides for generation of unique protein profiles useful in diagnosing, staging, guiding treatment, and monitoring disease progression and treatment success, as well as screening of agents to identify treatments of heart failure.

As used herein, “staging” refers to characterization of the progression and/or stage of heart failure according to the state of a protein or proteins of the profile, by comparing the state of the protein(s) in the profile from a patient or sample to the state of the protein(s) in the profile in appropriate controls, i.e healthy controls or diseased controls, at various stages along the progression of heart failure.

By “profile” as used herein it is meant to encompass selection as well as isolation and/or display of a state or states of a protein or proteins from a biological sample which is/are altered in heart failure. Proteins of the profile are present in an altered state in heart failure as compared to their state in healthy controls or a similar state as compared to their state in diseased controls for stages of heart failure. In one embodiment of the present invention, the profile may comprise a state of a single protein at a single time point. In another embodiment of the present invention, the profile may comprise states of two or more proteins at a single time point. In another embodiment of the present invention, the profile may comprise a state of a single protein at two or more time points. In yet another embodiment, the profile may comprisestates of two or more proteins at two or more time points.

By use of the term “protein” herein it is meant to include both the mature protein, and where appropriate, precursors of the mature protein, as well as isoforms and modified forms of the protein (e.g. post-translational modifications).

For purposes of the present invention by “state” of a protein it is meant to be inclusive of the presence, absence, level (e.g. quantity, or abundance), activity, tissue localization and/or intracellular localization of the protein, and/or a modification or change in modification to the protein including, but not limited to post-translational modifications, degradation products (e.g. fragments) and proteolysis or proteolytic cleavage and/or a change in interaction of the protein or modification thereof with other proteins as compared to the same protein in a healthy control, or for purposes of staging, a diseased control. In a preferred embodiment, the “state” comprises the level or activity of a protein in a subject as compared to the level or activity of the same protein in a healthy control or, for purposes of staging, a diseased control.

The state of a protein or proteins can be assessed in accordance with methods well known and used routinely by those of skill in the art. Examples of such methods include those taught herein for proteomic analysis by subfractionation followed by one-dimensional gel electrophoresis, two-dimensional gel electrophoresis, western blotting and/or silver staining. Other well known methods which can be used routinely to measure states of proteins of the profile of the present invention include, but are not limited, immunoassays, for example, ELISAs and radioimmunoassays, chromatographic separation techniques, for example, affinity chromatography, mass spectrometry, microfluidics, etc.

Exemplary post-translational modifications for purposes of the present invention include, but are not limited to, phosphorylation, oxidation, glycosylation, glycation, myristylation, phenylation, acetylation, nitrosylation, s-glutathiolation, amidation, biotinylation, c-mannosylation, flavinylation, farnesylation, formylation, geranyl-geranylation, hydroxylation, lipoylation, methylation, palmitoylation, sulphation, gamma-carboxyglutamic acids, N-acyl diglyceride (tripalmitate), O-GlcNAc, pyridoxal phosphate, phospho-pantetheine, and pyrrolidone carboxylic acid, and acylation. Another exemplary post-translational modification is proteolytic cleavage or proteolysis.

By “altered state” it is meant any measurable change or difference in the state of a protein as compared to the state of the same protein in a healthy control. For some proteins, no or very low levels of the protein may be present in a healthy control. For other proteins, detectable levels may be present normally in a healthy control. Thus, “altered state” further contemplates a level that is significantly different from the level found in a control. The term “significantly” refers to statistical significance. However, a significant difference between levels of proteins depends on the sensitivity of the assay employed, and must be taken into account for each protein assay.

By “healthy control” as used herein, it is meant a biological sample obtained from a subject known not to be suffering from heart failure, a sample obtained from the subject prior to the onset or suspicion of heart failure, or a standard from data obtained from a data bank corresponding to currently accepted normal states of these proteins. In animal models such as the swine model used herein, a healthy control is a SHAM-operated animal.

By “diseased control” as used herein, it is meant a biological sample obtained from a subject known to be suffering from heart failure, and more preferably suffering from a known stage of heart failure, e.g. presymptomatic or end-stage, or a standard from data obtained from a data bank corresponding to diseased states of these proteins in subjects suffering from heart failure. In animal models such as the swine model used herein, the diseased control is the LAD-ligated animal at a selected stage of heart failure.

By “subject” as used herein, it is meant a mammal, preferably a human.

Proteins of this profile for heart failure were identified using either whole tissue and/or fractions of whole tissue enriched for specific subproteomes including, for example, cytoplasmic, myofilament and mitochondrial proteins of ventricles of humans suffering from ischemic heart failure and healthy controls (non-diseased) as well as from the above-mentioned swine model of ischemic heart failure with its SHAM-operated controls.

Proteomic analysis of the human myocardium was conducted by two-dimensional gel electrophoresis (2-DE) on proteins from left ventricular tissue obtained from control (n=5) and end-stage ischemia-induced failing hearts (n=5). Optimization of both whole tissue extraction (see Example 1) and isoelectric focusing buffer using a zwitterionic detergent (see Example 2) allowed for increased membrane/hydrophobic protein detection. It will be appreciated that proteomic analysis and creation of a protein profile(s) may be conducted using other methodologies. These include, but are not limited to, protein chips and chromatography with an identification technique (e.g., HPLC or 2-D liquid chromatography with an identification technique such as, for example, mass spectrometry).

Using disclosed methods in Examples 7 to 11, proteins have been identified belonging to various subcellular regions including the cytoplasm (6-phosphofructokinase), myofilament (troponin T), cytoskeleton (vinculin), mitochondria (cytochrome C oxidase VA) and endoplasmic reticulum (GRP78). Eighteen of these proteins showed altered states between failing and normal human hearts. In particular, the abundance of glycogen phosphorylase, hUNC45, long chain fatty acid CoA ligase 1, TnC and 6-phosphofructokinase was increased in human ischemic heart failure. The abundance of alpha-actinin, aspartate aminotransferase, ATP synthase alpha chain, dihydrolipoamide dehydrogenase, GRP78, desmin, alpha-enolase, fructo-biphosphate aldolase A, moesin, NADH ubiquinone oxidoreductase 51 kDa, fumarate hydratase and TnT was decreased. In addition, (meta)vinculin exhibited a shift toward its isoform vinculin and novel enoyl CoA isomerase was found to be shifted towards its acidic form. Also see Tables 1 and 2.

In order to investigate the protein changes underlying the pathology of development and progression of heart failure, an ischemia induced heart failure model in swine was developed. Swine is an excellent choice of animal model for human cardiovascular diseases due to the similarities of both cardiovascular system and physiology between man and swine. In the described model (see Example 3), the left anterior descending coronary artery (LAD) was ligated (LAD swine), whereupon animals experienced a myocardial infarction to the apex of the left ventricle, the interventricular septum and the distal anterior right ventricle. Sham-operated swine (SHAM) underwent the same surgical procedure except the LAD was not occluded. Following surgery the animals were allowed to recover.

In the LAD swine, the myocardium in the infarcted area necrosed and could no longer generate muscle force to pump blood. The remaining viable parts of the heart had to compensate for the lost muscle mass. Those parts underwent diverse changes at the protein level in order to compensate for the loss of function in the infarcted region. As a result of the injury, the animals developed heart failure. After 4 weeks, myocardial function (left ventricular ejection fraction), measured by echocardiogram, was significantly decreased (39% for LAD vs 65% for SHAM). Chronic, end-stage heart failure was reached at about 6 weeks post-surgery.

This model allows for the termination of the animals at any chosen time point in the progression of the disease. Tissue samples from the viable parts of the left ventricle and blood samples were obtained before LAD occlusion and across the development of heart failure (samples from sham operated animals were obtained at respective time points). Samples from diseased and SHAM animals were studied to identify new protein markers for diagnosis, monitoring and staging of heart failure and new targets for therapeutic intervention (see Examples 4 to 11). Specifically, the inventors have identified reduced abundances of annexin V, cytochrome C oxidase VA, desmin, elastase IIIB, GRP 78, ventricular myosin light chain 1, myosin light chain 2, stathmin 3, T-complex protein 1 theta subunit, tropomyosin alpha 1, tropomyosin beta, TnT, and vimentin in LAD-ligated animals as compared to SHAM animals. The inventors identified increased abundances of 14-3-3 protein gamma, 2-oxoisovalerate dehydrogenase beta, beta lactoglobulin 1A and 1C, chloride intracellular channel protein 1, cytochrome b5, F-actin capping protein beta 1, HSP 27, MTCBP-1, NADH ubiquinone oxidoreductase 30 kDa subunit, NADH ubiquinone oxidoreductase 75 kDa subunit, peroxiredoxin 4, proliferating cell nuclear antigen, protein disulphide isomerase, TnI and TnC in LAD-ligated animals as compared to SHAM animals. In addition, the inventors identified a shift in tubulin alpha and tubulin beta towards the myofilament fraction of the myocytes, a shift in heat shock protein HSP 90-alpha to its acidic form, and a decrease in the degraded form of MRP 1 in LAD-ligated animals as compared to SHAM animals. Also see Tables 1 and 2.

TABLE 1
Proteins with changes in total abundance

| Name | pI/MW | Swine | Human |
|---|---|---|---|
| 14-3-3 protein gamma | 4.8/30000 | up | n.a. |
| 2-oxoisovalerate dehydrogenase | 5.6/40000 | up | n.a. |
| beta | | | |
| 6-phosphofructokinase | 8.5/80000 | n.a. | up |
| alpha-actinin | 5.5/100000 | n.a. | down |
| alpha-enolase | 6.7/45000 | n.a. | down |
| annexin V | 4.95/35000 | down | n.a. |
| aspartate aminotransferase | 7.0/39000 | n.a. | down |
| ATP synthase alpha chain | 8.3/55000 | n.a. | down |
| beta-lactoglobulin 1A and 1C | 4.3/19500 | up | n.a. |
| chloride intracellular channel | 5.4/31000 | up | n.a. |
| protein 1 | | | |
| cytochrome b5 | 5.0/17000 | up | n.a. |
| cytochrome C oxidase VA | 5.2/23000 | down | n.a. |
| Desmin | 5.8/58000 | down | down |
| dihydrolipoamide dehydrogenase | 6.9/60000 | n.a. | down |
| elastase IIIB | 5.65/23000 | down | n.a. |
| F-actin capping protein beta 1 | 5.5/34000 | up | n.a. |
| fructose-bisphosphate aldolase A | 8.4/40000 | n.a. | down |
| fumarate hydratase | 6.9/42000 | n.a. | down |
| glycogen phosphorylase | 6.75/85000 | n.a. | up |
| GRP 78 | 5.5/65000 | down | down |
| HSP 27 | 5.9/27000 | up | n.a. |
| hUNC 45 | 8.2/90000 | n.a. | up |
| long chain fatty acid CoA | 7.0/72000 | n.a. | up |
| ligase 1 | | | |
| MTCBP-1, | 5.4/21000 | up | n.a. |
| Moesin | 6.0/68000 | n.a. | down |
| ventricular myosin light chain 1 | 5.0/20000 | down | n.a. |
| myosin light chain 2 | 5.2/23000 | down | n.a. |
| NADH ubiquinone oxidoreductase | 6.2/30000 | up | n.a. |
| 30 kDa | | | |
| NADH ubiquinone oxidoreductase | 7.8/48000 | n.a. | down |
| 51 kDa | | | |
| NADH ubiquinone oxidoreductase | 5.35/70000 | up | n.a. |
| 75 kDa | | | |
| peroxiredoxin 4 | 6.1/28000 | up | n.a. |
| proliferating cell nuclear | 4.7/36000 | up | n.a. |
| antigen | | | |
| protein disulphide isomerase | 4.85/57000 | up | n.a. |
| stathmin 3 | 6.4/21000 | down | n.a. |
| T-complex protein 1 theta | 5.8/62000 | down | n.a. |
| subunit | | | |
| tropomyosin alpha 1 | 4.8/37000 | down | n.a. |
| tropomyosin beta | 4.7/38000 | down | n.a. |
| troponin C | (1D PAGE) | up | up |
| troponin I | (1D PAGE) | up | n.a. |
| troponin T | 5.35/39000 | down | down |
| Vimentin | 5.8/57000 | down | n.a. |

Note:
n.a. = data not available

TABLE 2
Proteins with changes in cellular localization,
ratios of PTM or isoforms

| Name | pI/MW | Swine | Human |
|---|---|---|---|
| HSP 90 alpha | 4.7/85000 | shift | n.a. |
| | | towards | |
| | | acidic form | |
| (meta)vinculin | 6.3/110000 | n.a. | shift towards |
| | | | metavinculin |
| MRP 1 | 5.9/85000 | decrease in | n.a. |
| | | degraded | |
| | | form | |
| novel enoyl CoA | 6.35/30000 | n.a. | shift towards |
| isomerase | | | acidic form |
| tubulin alpha | 4.95/50000 | shift to | n.a. |
| | | myo-filament | |
| | | fraction | |
| tubulin beta | 4.95/50000 | shift to | n.a. |
| | | myo-filament | |
| | | fraction | |

Note:
n.a. = data not available

The proteins identified as having altered states in human and swine heart failure include proteins involved in cellular organization, metabolic proteins, heat shock proteins and chaperones, protease and additional miscellaneous proteins.

Cell organization proteins comprise a group of proteins involved in structural organization of the cell and cellular integrity. This group of proteins can be divided into cytoskeletal/intermediate and contractile proteins as well as proteins that regulate structural organization of these subproteomes within the cell.

Cytoskeletal/intermediate proteins identified by the inventors herein as being in an altered state in heart failure include alpha-actinin, desmin, tubulin alpha, tubulin beta, vimentin, (meta)vinculin, moesin and MTCBP-1.

For example, a shift in (meta)vinculin towards metavinculin was identified herein to be increased in heart failure. Metavinculin is a muscle specific protein and is different in length from vinculin by 68 amino acids inserted near the C-terminus. Vinculin as a splice variant of (meta)vinculin. This protein is believed to be involved in cell adhesion through the attachment of the microfilaments to the plasma membrane. Vinculin has been shown to be increased in human dilated cardiomyopathy that was accompanied by cardiomyocyte remodelling and cytoskeletal abnormalities (Heling A et al. Circ. Res. 2000 86:846-53). In a case of human idiopathic dilated cardiomyopathy metavinculin was decreased, while the expression of vinculin was unchanged (Maeda M et al. Circulation 1997 95:17-20).

The observation described herein is indicative of the ratio of vinculin and metavinculin alone or in combination with other proteins described herein to be useful for monitoring, diagnosing, staging, evaluating treatments and treating heart failure.

Levels of the cytoskeletal proteins alpha-actinin, moesin, desmin, tubulin alpha and beta and vimentin were found by the inventors to be decreased in heart failure.

Alpha-actinin, also known as a-actinin skeletal muscle isoform 2 and F-actin cross-linking protein, is expressed in both skeletal and cardiac muscle and is located at the actin filament. This protein is believed to act as a bundling protein to anchor F-actin to different intracellular structures. It is composed of an actin-binding domain, 2 calponin-homology domains, as well as 2 EF-hand Ca²⁺ binding domains.

Moesin, also known as membrane-organizing extension spike protein, is another cytoskeletal protein demonstrated by the inventors herein to be decreased during heart failure. Moesin is a member of the ezrin-radixion-moesin family of proteins which act as regulatory molecules linking F-actin to major cytoskeletal structures of the plasma membrane. This protein plays a key role in the control of cell morphology, adhesion, and motility.

As shown by the inventors herein, levels of desmin were also decreased in heart failure in both humans and swine. Desmin is an intermediate filament in muscle cells which functions by connecting myofibrils to each other as well as to the plasma membrane. Defects in desmin are known to result in familial cardiac and skeletal myopathy (CSM). Patients with CSM exhibit skeletal muscle weakness, cardiac conduction blocks, arrhythmias and restrictive heart failure. Desmin null-mice develop concentric cardiomyocyte hypertrophy. This type of hypertrophy was accompanied by induction of embryonic gene expression and later by ventricular dilatation, and compromised systolic function (Milner D J et al. J. Mol. Cell. Cardiol. 1999 31:2063-76).

The inventors also identified vimentin as having decreased levels during heart failure. Vimentins are class-III intermediate filaments found in various non-epithelial cells, especially mesenchymal cells. Vimentin has been shown to be decreased in hypoxia-induced right ventricular hypertrophy in bovids (Lemler et al. Am. J. Physiol. Heart Circ. Physiol. 2000 279:H1365-76). This decrease, together with a decrease in other important proteins in the cytoskeleton, has been associated with an increase of myocyte stiffness, disruption of the normal myocardial cytoskeleton and contractile dysfunction.

Tubulin alpha and tubulin beta were also identified by the inventors as being decreased in heart failure. Tubulin is the major constituent of microtubules in eukaryote cells. It binds ATP on its alpha chain and GTP both at an exchangeable site on its beta chain and at a non-exchangeable site on the alpha chain. The highly acidic carboxyl-terminal region may bind cations such as calcium. Increased tubulin polymerization and microtubule density has been observed in left ventricle of hypertrophic and failed hearts in both animal models and humans (Tagawa H et al. Circ. Res. 1998 82:751-61, Aquila-Pastir et al. J. Mol. Cell Cardiol. 2002 34:1513-23). The increase in microtubule density was accompanied by a shift of tubulin from the soluble to the insoluble fraction in hypoxia induced right ventricular hypertrophy in bovids (Lemler W S et al. Am. J. Physiol. Heart Circ. Physiol. 2000 279:H1365-76) and in a feline model of pressure overload induced right ventricular heart failure (Tagawa H et al. Circulation,1996,93:1230-43). The increase in microtubule density is associated with an increase in myocyte stiffness and contractile dysfunction. Overall, a disruption of the normal myocardial cytoskeleton is observed (decreased or absent striation). The shift of tubulin from the cytosolic towards the insoluble fraction increases stress in the myocyte. Stretching of the myocyte upon increased density of microtubules leads to increased Ca²⁺ influx via mechanosensitive L-type Ca²⁺ channels. This can be seen initially as a compensatory mechanism to increase available Ca²⁺ for contraction. Over time, however, the extra Ca²⁺ in the cytosol leads to diastolic dysfunction.

It is believed that the down-regulation and/or redistribution to the cytosol of proteins of the sarcomeric skeleton including, but not limited to vinculin/metavinculin, alpha-actinin, moesin, vimentin, desmin and tubulins in heart failure contributes to the observed contractile dysfunction.

Levels of another cytoskeleton-related protein, membrane-type 1 matrix metalloproteinase cytoplasmic tail-binding protein (MTCBP-1), were found to be increased in heart failure. MTCBP-1 is a 19-kDa protein that belongs to the newly proposed cupin superfamily composed of proteins with diverse functions (Uekita T. et al. J Biol Chem. 2004 279:12734-43). MTCBP-1 expressed in cells forms a complex with membrane-type 1 matrix metalloproteinase (MT1-MMP) and co-localizes with alpha-actinin at the sarcolemma of cardiomyocytes. These characteristics are consistent with the location of costameres and the modulation of local myocyte adhesion to the extracellular matrix thereby. MT1-MMP is secreted in a proenzyme form and requires proteolytic cleavage for activation. Active MT1-MMP is then inserted into the plasma membrane facing the extracellular space, where it can cleave pericellular substrates. MT1-MMP is a zinc-dependent endopeptidase consisting of a catalytic, hinge, hemopexin-like, transmembrane domain and a cytoplasmic tail (Osenkowski P. et al. J Cell Physiol. 2004 200:2-10). This protein is regulated by function of the cytoplasmic tail. Internalization of MT1-MMP into the membrane from the surface depends on the sequence of its cytoplasmic tail (Uekita T et al. J Cell Biol. 2001 155:1345-56). Mutations at the cytoplasmic tail lead to the accumulation of inactive enzyme at the adherent edge. The cytoplasmic tail of MT1-MMP also interacts with intracellular regulatory proteins, which modulate translocations of the protease across the cell to the leading edge of the migrating cell. MTCBP-1 is one of the modulators of MT1-MMP and therefore may be involved in the balance of matrix synthesis/turnover known as LV remodeling in heart failure.

Contractile proteins identified herein as having an altered state in heart failure include ventricular myosin light chain 1, myosin light chain 2, TnT, TnI, TnC and tropomyosin alpha 1 and tropomyosin beta.

For example, ventricular myosin light chain 1, also known as MLC-1v, myosin alkali light chain 3, or essential myosin light chain, was found to be decreased in heart failure. MLC-1v is involved in Ca²⁺ dependent regulation of muscle contraction. Mutations of MLC-1v have been found in patients with idiopathic hypertrophic cardiomyopathy (Morano I. J Mol Med. 1999 77:544-55). Due to its essential role in muscle contraction, down-regulation of MLC-1 will impair contractility of the myocytes and decrease cardiac function in heart failure.

Levels of myosin regulatory light chain 2, also referred to as MLC-2, regulatory MLC, and phosphorylatable MLC, were also identified as being decreased in heart failure. Myosin light chain 2 is involved in Ca²⁺ dependent regulation of muscle contraction. This protein slows down the rate of tension development of myosin. Myosin light chain 2 is important for myosin structure and function. Mutations of myosin light chain 2 have been found in patients with hypertrophic cardiomyopathy (Flavigny J et al. J. Mol. Med. 1998 76:208-14). Removal of myosin light chain 2 changes the structure of the cardiac myosin molecule and reduces Vₘₐₓ and shortens velocity in skeletal muscle (Morano et al. J. Mol. Med. 1999 77:544-55). Removal of myosin light chain 2 also eliminates Ca²⁺ dependency of rate of force redevelopment of cross-bridges. This indicates down-regulation of the attachment rate constant of cross-bridges by myosin light chain 2. Increased attachment-rate constant of cross-bridges leads to increased force generating cross-bridges at a given Ca²⁺ level and consequently to increased stiffness and Ca²⁺ sensitivity. A decrease in MLC-2 levels in dilated cardiomyopathy in humans has been reported (Margossian S S et al. Circulation. 1992 85:1720-33). However, the inventors are not aware of any reports on changes in myosin light chain 2 levels in ischemic cardiomyopathy. The down-regulation of myosin light chain 2, together with an increase in intracellular Ca²⁺ during the progression of heart failure, leads to increased tension and consequently diastolic dysfunction.

Tropomyosin alpha 1, also referred to as alpha-tropomyosin, was also identified by the inventors as having decreased levels in heart failure. Tropomyosin alpha 1 binds to actin filaments in muscle and non-muscle cells. This protein plays a central role in association with the troponin complex and in the calcium dependent regulation of vertebrate striated muscle contraction (Solaro R J et al. Circ. Res. 1998 83:471-80). Smooth muscle contraction is regulated by the interaction of this protein with caldesmon. In non-muscle cells tropomyosin 1 alpha chain is implicated in stabilizing cytoskeleton actin filaments.

Tropomyosin beta, also referred to as beta-tropomyosin or tropomyosin 2, was also identified by the inventors as having decreased levels in heart failure. This protein binds to actin filaments in muscle and non-muscle cells. Tropomyosin beta plays a central role, in association with the troponin complex, in the calcium dependent regulation of vertebrate striated muscle contraction (Solaro R J et al. Circ. Res. 1998 83:471-80). Smooth muscle contraction is regulated by its interaction with caldesmon. In non-muscle cells tropomyosin beta is implicated in stabilizing cytoskeleton actin filaments.

Troponin T, also know as troponin T2 or TnT, was also identified by the inventors as having decreased levels in heart failure in both humans and swine. TnT binds the troponin complex to tropomyosin. With tropomyosin the protein mediates contraction of vertebrate striated muscle in response to calcium (Solaro R J et al. Circ. Res. 1998 83:471-80).

Down-regulation of contractile proteins MLC-1, MLC-2 tropomyosin alpha and beta, and TnT in heart failure observed by the inventors supports impaired contractility of the myocytes and decreased cardiac function.

Proteins involved in structural organization identified herein as having an altered state in heart failure include F-actin capping protein beta 1, HSP 27, HSP 90 alpha, hUNC 45 (human striated muscle UNC 45), MRP 1 (conjugate export pump protein), stathmin 3 and T-complex protein 1 theta subunit.

For example, the inventors found an increase in F-actin capping protein beta 1, also known as CapZ beta 1 or CP-β₁, in heart failure. This protein is part of a heterodimer of an alpha and a beta subunit and binds in a Ca²⁺-independent manner to the fast growing ends of actin filaments thereby blocking the exchange of subunits at these ends. Unlike other capping proteins (such as gelsolin and severin), this protein does not sever actin filaments (Caldwell J E et al. Biochemistry. 1989 28:8506-14). Beta subunit isoforms 1 and 2 differ in their C-termini and have different locations within the cell. Beta 1 is localized at the Z-lines, beta 2 at the intercalated discs (Schafer D A et al. J Cell Biol. 1994 127:453-65). CP-β₁ caps the barbed ends of the thin filaments and anchors them to the Z-line, which is critical for normal muscle development (Schafer D A et al. J. Cell. Biol. 1995,128:61-70) (Pyle W G et al. Circ. Res. 2002 90:1299-306). Disruption of actin-CP-β₁ interaction impairs myofibrillogenesis and produces gross myofibrillar disarray (Schafer D A et al. J. Cell. Biol. 1995 128:61-70). Since CP-β₁ can nucleate filament formation in vitro, it may also do so in vivo during myofibrillogenesis and thus may induce formation of new myofilaments. Substitution of CP-β₁ by CP-β₂ in the mouse leads to cardiac hypertrophy and high death rates after a few weeks. These mice also exhibit increased myofilament Ca²⁺ sensitivity and disrupted PKC-dependent myofilament regulation (Pyle W G et al. Circ. Res. 2002 90:1299-306). Overexpression of CP-β₂ for CP-β₁ produces altered morphology of the intercalated discs without increased lethality and myofibril disruption (Hart M C et al. J Cell Biol. 1999 147:1287-98). The inventors believe that increases in CP-β₁ lead to an increase in myofilament formation as a compensatory mechanism in the early stages of heart failure development.

Heat shock protein 27, also known as HSP 27, stress-responsive protein 27, SRP27, estrogen-regulated 24 kDa protein, and 28 kDa heat shock protein, was found to be increased in heart failure. It has also been found to be increased in human dilated cardiomyopathy (Knowlton A A et al. J. Mol. Cardiol. 1998 30:811-8). HSP 27 is involved in stress resistance and actin organization as well as in protection against apoptosis involving the cytochrome c pathway. Cytochrome c binds to Apaf-1 and triggers its oligomerisation. This complex then attracts the inactive unprocessed pro-form of the proteolytic enzyme caspase-9 which is thereafter activated and initiates the apoptotic process. HSP 27 binds to cytochrome c and prevents it binding to Apaf-1 (Latchman D S et al. Cardiovasc Res. 2001 51:637-46). Overexpression of HSP 27 protects the integrity of microtubules and the actin cytoskeleton of endothelial cells exposed to ischemia (Loktionova S A et al. Am J Physiol. 1998 275:H2147-58). HSP 27 has been shown to bind to eNOS and stimulates its activity. The increased synthesis of nitric oxide by eNOS is a potent mechanism against oxidative stress (Tanonaka K et al. Biochem Biophys Res Commun. 2001 283:520-5). Increase in HSP 27 in heart failure is therefore viewed by the inventors as a compensatory mechanism that leads to increased protection of cardiomyocytes against apoptotic stimulus and oxidative stress during heart failure.

The inventors also identified heat shock protein HSP 90 alpha, also referred to as HSP86, as shifting to an acidic form in heart failure. This protein is also involved in cell organization, and more specifically in structural organization. This protein functions as a molecular chaperone exhibiting ATPase activity and being involved in maintenance of proteins such as steroid receptors. Overexpression of several members of the heat shock protein family is known to protect organs, including the heart, from endogenous and exogenous stresses. The phosphorylation of HSP 90 alpha is linked to its chaperoning function. HSP 90 alpha is involved in protection against apoptosis involving the cytochrome c pathway. Cytochrome c binds to Apaf-1 and triggers its oligomerization. This complex then attracts the inactive unprocessed pro-form of the proteolytic enzyme caspase-9, which is thereafter activated and initiates the apoptotic process. HSP 90 alpha binds to Apaf-1 and prevents it binding to cytochrome c. HSP 90 alpha has also been shown to bind to eNOS and stimulates its activity. The increased synthesis of nitric oxide by eNOS is a potent mechanism against oxidative stress. The inventors believe that the shift towards an acidic form of HSP 90 alpha together with the increase in HSP 27 in heart failure is a compensatory mechanism that leads to increased protection of cardiomyocytes against apoptotic stimulus and oxidative stress during heart failure.

The striated muscle human homolog of UNC-45, referred to herein as hUNC-45, was identified herein for the first time in human cardiac tissue, and exhibited increased levels during heart failure. UNC-45 from C. elegans is a member of the canonical UCS protein family and was one of the earliest molecules to be shown genetically to be necessary for sarcomere assembly. UNC-45 from C. elegans has also been shown to function as a chaperone by binding the proteins HSP 90 and myosin (Barral J M et al. Science 2002 295:669-71). It has recently been reported that whereas C. elegans, S. cerevisiae and other lower organisms possess a single UCS protein, both mice and humans possess two distinct isoforms of UCS proteins (based on genetic screening) (Price M G et al. J Cell Sci,2002,115:4013-23). These proteins are termed the striated muscle and general cell isoforms based on their tissue localization in mice. Utilizing anti-sense mRNA to abrogate gene transcription in isolated cells, it has been reported that decreasing the general cell isoform mRNA reduces cell proliferation and fusion. Conversely, decreasing the striated muscle isoform mRNA was observed to affect cell fusion and sarcomere organization. All of the previous research reported to date has been on mice and/or isolated cell models and the inventors are not aware of any published observations of the protein product of UNC-45 in humans. It is the inventors' belief that an increase in hUNC-45 may be indicative of cardiomyocyte remodeling during heart failure.

The inventors also found levels of a modified (degraded) form of conjugate export pump protein to be decreased in heart failure. This protein is also referred to as MRP 1. MRP 1 is a member of the superfamily of ATP-binding cassette membrane transporters. Activation and over-expression of MRP 1 causes multi-drug resistance in several tumor lines. MRP 1 binds and removes drugs, toxins and glutathione from the intracellular compartment to the extracellular space. In a MRP 1 deficient mouse model, the lack of MRP 1 expression in tissues normally expressing high levels of MRP 1 (for example heart) leads to an increase (˜45% in the heart) of glutathione protein levels (Lorico et al. Cancer Res. 1997 57:5238-42). Glutathione is an important oxygen radical scavenger. The decrease of active MRP1 due to degradation is believed to decrease glutathione concentration in myocytes, partly impairing protection of cardiomyocytes against increased oxidative damage during heart failure.

Stathmin 3, also referred to as SCG10-like protein and oncoprotein 18, was also identified by the inventors as having decreased levels in heart failure. Stathmin 3 sequesters unpolymerized tubulin subunits (1:2 molar ratio) to maintain a subunit pool substantially higher than the critical concentration of microtubules. Stathmin 3 decreases the effective concentration of tubulin subunits for polymerization and prevents uncontrolled cell growth. In isolated cells, lack of stathmin 3 causes cell elongation. The growth of microtubules at the positive end is balanced by depolarization (dependent on GTP hydrolysis) at the negative end (Mistry S J et al. Mt Sinai J Med 2002 69:299-304). The down-regulation of stathmin 3 in heart failure upsets the dynamic equilibrium of microtubule formation. The increase of microtubule density is associated with an increase of myocyte stiffness and contractile dysfunction. Stretching of the myocyte upon increased density of microtubules leads to increased Ca²⁺ influx via mechanosensitive L-type Ca²⁺ channels. Initially, this can be seen as a compensatory mechanism to increase available Ca²⁺ for contraction. Over time, however, the extra Ca²⁺ in the cytosol leads to diastolic dysfunction. The decrease of stathmin 3 is also consistent with the shift of tubulin alpha and beta towards the myofilament fraction of the cardiomyocytes.

While not wishing to be bound to any particular theory, it is the inventors' belief that decreased levels of cytoskeletal proteins including tubulin, vimentin and their structural partner stathmin has a direct impact on the increase of microtubule density and consequently the increase in myocyte stiffness and contractile dysfunction.

T-complex protein 1 theta subunit, also known as TCP-1-theta or CCT-theta, was found to be decreased in heart failure as well. TCP-1-theta is a molecular chaperone and assists in the folding of proteins upon ATP hydrolysis. This protein also has a role, in vitro and in vivo, in the folding of actin, tubulin and myosin II heavy chain (Dunn A Y et al. J Struct Biol. 2001 135:176-84). TCP-1-theta is part of a hetero-oligomeric complex of about 850 to 900 kDa that forms two stacked rings, 12 to 16 nm in diameter. Mutations to TCP-1 subunits can result in global cytoskeletal disorganization resulting from actin and tubulin misfolding (Vinh D B N et al. Proc Natl Acad Sci. 1994 91:9116-20). Down-regulation of TCP-1-theta destabilizes the cytoskeletal structure of cardiomyocytes contributing to contractile dysfunction during heart failure.

The altered states of structural proteins identified by inventors in heart failure compliment each other in their effect on overall stability of cardiomyocytes and therefore become part of a profile of protein useful in diagnosing, monitoring, staging, evaluating treatments and treating heart failure.

Metabolic proteins comprise a group of proteins involved in regulation of energy supply through metabolic pathways including the TCA cycle, β oxidation, glycolysis and oxidative phosphorylation. Metabolic proteins identified herein as having an altered state in heart failure include 2-oxoisovalerate dehydrogenase beta, 6-phosphofructokinase, glycogen phosphorylase, alpha-enolase, dihydrolipoamide dehydrogenase, fructose-bisphosphate aldolase A, cytochrome b5, cytochrome C oxidase VA, ATP synthsa alpha chain, fumarate hydratase, NADH ubiquinone oxidoreductase 30 kDa subunit, NADH ubiquinone oxidoreductase 51 kDa subunit, NADH ubiquinone oxidoreductase 75 kDa subunit, aspartate aminotransferase, long chain fatty acid CoA ligase 1 and novel enoyl CoA isomerase. A protein profile of altered states of one or more of these proteins is believed to be indicative of a shift of cardiac cells from lipid metabolism to carbohydrate or amino acid metabolism as an oxygen conserving means in energy production.

For example, the inventors found 2-oxoisovalerate dehydrogenase beta to be increased in heart failure. Other names for this protein include branched-chain alpha-keto acid dehydrogenase E1 component beta chain, BCKDH E1-beta and alpha-keto-beta-methylvalerate dehydrogenase. The branched-chain alpha-keto dehydrogenase complex is the rate-limiting enzyme in the catabolism of branched-chain amino acids (valine, leucine and isoleucine) to acetyl-CoA and succinyl-CoA. It contains multiple copies of three enzymatic components: branched-chain alpha-keto acid decarboxylase (E1), lipoamide acyltransferase (E2) and lipoamide dehydrogenase (E3). This protein forms a heterodimer of an alpha and a beta chain. The perfusion of rat hearts with 2-oxoisocaproate (substrate for BCKDH in leucine catabolism) leads to a reduction in beta-oxidation of fatty acids, while 2-oxoisovalerate (substrate for BCKDH in valine catabolism) does not. In contrast to valine that is only partly metabolized (5 ATP) in the heart, leucine is fully oxidized in the heart, yielding 39 ATP/molecule (Letto J et al. Biochem Cell Biol. 1990 68:260-65). This can be seen as an alternative means of energy production in the heart, since acetylacetate (from leucine catabolisms), together with succinyl-CoA (from, for example, isoleucine catabolism) form acetoacetyl-CoA, a potent inhibitor of beta-oxidation. Fatty acid oxidation is decreased by as much as 40% in hypertrophied hearts (rat) leading to recruitment of other energy sources (Leong H S et al. Comp Biochem Physiol A Mol Integr Physiol. 2003 135:499-513). Increased glucose and amino acid oxidation is the consequence. Increased amino acid oxidation, however, leads eventually to an increase in ammonium, which again will drain pyruvate from the citric acid cycle, reducing the contribution of glucose in the overall energy production (Wagenmarkers A J M et al. Int J Sports Med. 1990 11:S101-13). Reduced glucose oxidation has been found in hypertrophied hearts (Leong H S et al. Comp Biochem Physiol A Mol Integr Physiol. 2003 135:499-513). The inventors believe that the increase of branched-chain amino acid catabolism and the subsequent reduction in glucose metabolism was suggested to be responsible in part for the impairment of oxidative metabolism in healthy subjects during prolonged exercise (Wagenmarkers A J M et al. Int J Sports Med. 1990 11:S101-13). The increase in BCKDH during heart failure indicates a switch from fatty acid to amino acid metabolism in the heart. The reduction in fatty acid oxidation reduces the 02 demand in the heart, but, in later stages of the disease may increase the ammonium concentration in the heart, reducing the amount of pyruvate (and other intermediates) to be fed into the citric acid cycle and reducing overall energy production in the heart.

Increased levels of 6-phosphofructokinase, also known as phosphofructokinase 1, phosphohexokinase, phosphofructo-1-kinase isozyme A, PFK-A, phosphofructokinase-M, and PFKM, were also identified by the inventors during heart failure. The function of 6-phosphofructokinase, muscle type, is to catalyze the reaction of ATP and D-fructose 6-phosphate to ADP and D-fructose 1,6-bisphosphate. 6-phosphofructokinase is one of the first enzymes in glycolysis and as such, controlling its activity results in an activation or inhibition of glucose metabolism. There are three distinct forms of phosphofructokinase with PFKM being located in muscle tissue, PFKL being located in the liver and PFKP being located in the platelets. Specifically, phosphofructokinase is a tetramer with muscle possessing a homotetramer of PFKM, liver possessing a homotetramer of PFKL, and red blood cells possessing a heterotetramer of M3L, M2L2, or ML3. In addition, due to alternative splicing, there are two isoforms of PFKM (P08237-1 and P08237-2) that differ by the addition of 31 amino acids in the middle and 28 amino acids at the C-terminus in isoform 1. Defects in PFKM are the cause of Tarui disease, in which patients are unable to perform short periods of intense activity. The inventors believe that the increase in 6-phosphofructokinase may be indicative of a switch from fatty acid to carbohydrate metabolism in the heart. A reduction in fatty acid oxidation reduces the 02 demand in the heart.

Increased levels of glycogen phosphorylase, also known as myophosphorylase, were also identified by the inventors during heart failure. Glycogen phosphorylase catalyzes the first step in glycogenolysis, (1,4-alpha-D-glucosyl)n+phosphate=(1,4-alpha-D-glucosyl)n-1+alpha-D-glucose 1-phosphate. Glycogenolysis is increased in the myocardium during oxygen deprivation (Neely J R et al. Am J Physiol. 1973 225:651-8) and glycogen phosphorylase activity is increased in patients with high-grade cardiac hypertrophy (Lehmann et al. Biomed Biochim Acta. 1987 46:S602-5). This protein is also found in the blood of patients with acute myocardial infarction (Mair J. Clin Chim Acta. 1998 272:79-86). Use of glycogen phosphorylase inhibitors has been suggested for the treatment of cardiovascular diseases such as ICM (Published U.S. patent application No. 2004082646 and WO 2004037233).

The increase in glycogen phosphorylase increases the amount of glucose for glycolysis, reducing the amount of oxygen required by the myocardium to produce energy and is, together with the increase in 6-phosphofructokinase, a further indication for a shift in cardiac metabolism away from fatty acids.

The basic form of a novel enoyl CoA isomerase was also identified by the inventors as having decreased levels in favor of its acidic form in heart failure. By MS/MS sequencing is was determined that the protein is one of two proteins, NCBI 16924265 or NCBI 15080016. The DNA sequence for 16924265 was submitted to the NIH Mammalian Gene Collection (MGC) database on Nov. 13, 2001 after screening of a cDNA library from melanotic melanoma skin cells. Similarly, the DNA sequence for 15080016 was submitted to the NIH MGC database on Jul. 30, 2001 after screening of a cDNA library from pancreatic epithelioid carcinomas. Thus, irrespective of which of these proteins is the actually present, this is the first evidence of this protein in cardiac tissue. A sequence similarity search conducted against other proteins in the NCBI and SWISS-PROT databases revealed that both of these proteins exhibit approximately 96% similarity to the human protein Δ^{3,5}-Δ^{2,4}-dienoyl-CoA isomerase (Accession # Q13011). This known protein is an auxiliary protein in the peroxisomal fatty acid oxidation pathway and functions by isomerizing the double bond from the 3,5 to the 2,4 location in unsaturated fatty acids. This new location of the double bond allows the fatty acid to enter the main peroxisomal beta oxidation pathway. The existence of the peroxisomal targeting sequence (FSKL) at the C-terminus of each of these novel proteins is indicative of these proteins being located in the peroxisome. Thus, it is believed that this protein may play an additional role in peroxisomal lipid metabolism. The shift in novel enoyl CoA isomerase towards its acidic form is another indicator for a shift in cardiac metabolism away from fatty acid oxidation during heart failure.

Levels of dihydrolipoamide dehydrogenase, also known as dihydrolipoyl dehydrogenase or glycine cleavage system L protein, were also shown by the inventors to be decreased in heart failure. This protein is a member of the pyruvate dehydrogenase (PDH) complex. The PDH complex catalyzes the rate-determining step in aerobic carbohydrate metabolism and catalyses the oxidative decarboxylation of pyruvate to form acetyl-CoA and entry into the TCA cycle. In a mouse model of hypertrophy, increased glycogenolysis and glycolysis without corresponding increases of glucose oxidation was observed (Wambolt R B et al., J. Mol. Cell. Cardiology. 1999 31:493-502).

The decrease in dihydrolipoamide dehydrogenase limits the amount of pyruvate that can be fed in to the citric acid cycle, reducing the amount of oxygen consumption by the myocardium during carbohydrate metabolism.

Levels of fructose-bisphosphate aldolase A, also known as muscle-type aldolase or fructoaldolase, were also shown by the inventors to be decreased in heart failure. This protein catalyzes the energy dependent aldol condensation within the glycolysis pathway.

A decrease in fructose-bisphosphate aldolase A leads to a decrease in energy production via glycolysis.

Levels of alpha-enolase, also known as 2-phospho-D-glycerate hydrolyase, non-neural enolase, NNE, enolase 1, or phosphopyruvate hydratase, were also shown by the inventors to be decreased in heart failure. Enolase catalyzes the reaction of 2-phospho-D-glycerate to phosphoenolpyruvate and H₂0 in the glycolysis pathway. Enolase exists as a dimer of either α/α or α/β enolase with α/α being the predominant fetal isoform and a transition to a mixed α/α and α/β isoform in adults. Isoform shifting effecting the β isoform and resulting in a return to the fetal isoform has been shown in a rat model of cardiac hypertrophy (Keller et al. Am. J. Physiol. 1995 269:H1843-51). It is the inventors' belief that the decrease in alpha-enolase in the heart may be indicative of a change in cardiac energy household during heart failure.

Aspartate aminotransferase, also known as transaminase A or glutamate oxaloacetate transaminase-1, levels were also identified by the inventors as decreasing during heart failure. This protein catalyzes the transamination of aspartate (and other amino acids) necessary to feed amino acids into the TCA cycle. Enzymatic activity of cytoplasmic aspartate aminotransferase is increased in human heart failure (Neely J R et al. Am J Physiol. 1973,225:651-8). Measurement of transaminase activity in blood was the first diagnostic biomarker for acute myocardial infarction (Ladue J S et al. Science. 1954 120:497-9).

A decrease in aspartate aminotransferase reduces the catabolism of aspartate (and other amino acids), further reducing the energy production in the failing heart.

Levels of long chain fatty acid CoA ligase 1, also known as palmitoyl-CoA ligase 1, were also shown by the inventors to be decreased in heart failure. Activation of long-chain fatty acids is required for synthesis of cellular lipids and fatty acid degradation via beta-oxidation. Long chain fatty acid CoA ligase 1 preferentially metabolizes palmitoleate, oleate and linoleate. Long-chain fatty acid oxidation is impaired in volume-overloaded rat hearts (Christian B et al. Mol Cell Biochem. 1998 180:117-28).

An increase in long chain fatty acid CoA ligase 1 indicates an increase in fatty acid metabolism in the failing heart.

The inventors expect that the methods of the invention can be carried out by obtaining a profile of one or more substrates (i.e., proteins) and/or one or more metabolites involved in at least one of the glycolytic pathway, the TCA cycle, the citric acid cycle, the beta oxidation pathway, and the electron transport chain. Preferably, the one or more substrates are selected from the above-mentioned enzymes, and the one or more metabolites are metabolites of those enzymes.

Several proteins responsible for energy and oxygen supply and distribution were found altered in failing hearts.

For example, cytochrome b5, an important member of the electron transport chain and a membrane bound hemoprotein which functions as an electron carrier for several membrane bound oxygenases, is shown herein to be increased in heart failure. In contrast, cytochrome b5 is decreased in pacing induced canine HF (Heinke M Y et al. Electrophoresis. 1999 20:2086-93). Metmyoglobin (metMb) reduction by metMb reductase from heart muscle requires cytochrome b5 as an electron-transfer mediator (Livingston D J et al. J Biol Chem. 1985 260:15699-707). Cytochrome b5 can receive electrons from NADPH-cytochrome P450 reductase and NADPH-cytochrome b5 reductase and can transfer them to acceptors like cytochrome c, cytochrome P450 or methemoglobin (Schenkman J B et al. Pharmacol Ther. 2003 97:139-52). Increases in cytochrome b5 enhance the reduction of metmyoglobin and therefore the release of oxygen in the heart muscle. Cytochrome b5 may also be part of an oxygen sensor and as such, a starting point of the cascade involving the hypoxia-inducible transcription factor 1 and von Hippel-Lindau tumor suppressor protein, that ultimately controls regulation of the erythropoietin gene (Zhu H et al. Nephrol Dial Transplant. 2002 17(suppl 1):3-7).

The increase in cytochrome b5 in heart failure could therefore allow the up-regulation of erythropoietin and the increase in red blood cells, the main carrier of oxygen in the mammalian body.

The inventors also identified ATP synthase alpha chain, also known as H+-transporting two-sector ATPase, as being decreased in heart failure. ATP synthase alpha chain is the regulatory subunit of ATP synthase (complex V) that produces ATP from ADP in the presence of a proton gradient across the membrane. ATP synthase activity is decreased in pacing induced canine HF, while no change in alpha subunit abundance was found (Marin-Garcia J et al. Cardiovasc Res. 2001 52:103-10).

Down-regulation of the regulatory subunit of complex V can cause an uncoupling of the respiratory chain and a reduction in energy production by the mitochondria.

The inventors also identified NADH-ubiquinone oxidoreductase 51 kDa subunit, also known as complex I-51 KD, as being decreased in heart failure. NADH-ubiquinone oxidoreductase 51 kDa subunit is part of the mitochondrial electron transport and oxidative phosphorylation system. Complex I activity is decreased with cardiomyopathies in humans and animal models. Oxygen free radicals have been shown to cause contractile failure and structural damage in the myocardium of the rat (Ide T et al. Circ Res,1999,85:357-63). In addition, a decrease in myocardial antioxidant reserve has been shown in heart failure. Levels of activity of complex I are decreased in human cardiomyopathy (Marin-Garcia J et al. J Inherit Metab Dis, 2000,23:625-33).

Cytochrome C oxidase VA levels were also identified by the inventors as decreasing during heart failure. Cytochrome C oxidase VA, also referred to as cytochrome-C oxidase polypeptide VA, complex IV (mitochondrial electron transport), NADH cytochrome c oxidase, or cytochrome a3, catalyzes the oxidation of four molecules of reduced cytochrome c in the intracristal (or intermembrane) space using one oxygen molecule and four protons from the mitochondrial matrix, producing two molecules of water, and lowering the concentration of protons in the mitochondrial matrix. Levels of activity of this protein have also been shown to be decreased in human cardiomyopathy (Marin-Garcia et al. J. Inher. Metab. Dis. 2000 23:625-33) and in transgenic heart failure mice expressing mutated myosin heavy chain (Lucas et al. Am. J. Physiol. Heart Circ. Physiol. 2003 284:H575-83).

A decrease of complex I during heart failure, together with the decrease of complexes IV and V, causes an uncoupling of the respiratory chain, the generation of oxygen radicals and a reduction in energy production by the mitochondria. Oxygen radicals will further damage the myocytes, while a lack of ATP will decrease myocardial contraction.

Levels of fumarate hydratase, also known as fumarase, were found by the inventors to be decreased in heart failure. Fumarase catalyzes the citric acid cycle reaction of (S)-malate to fumarate and H₂O. Additionally, fumarase is believed to function as a tumor suppressor. Through alternative splicing, two isoforms of fumarase exist. One form exists in the mitochondria and the other form is located in the cytoplasm. The disease fumaricaciduria, characterized by progressive encephalopathy, developmental delay, hypotonia, cerebral atrophy as well as lactic/pyruvic acidemia is due to a deficiency in fumarase. Defects in fumarase are also the cause of multiple cutaneous and uterine leiomyomata, an autosomal dominant condition characterized by benign smooth muscle tumors of the skin (and uterus in females).

NADH ubiquinone oxidoreductase 30 kDa subunit, also known as NADH dehydrogenase (ubiquinone) 30 kDa, complex I-30 KD or CI-30 KD, and NADH-ubiquinone oxidoreductase 75 kDa subunit, also referred to as NADH dehydrogenase (ubiquinone) 75 kDa, complex I-75 KD, coenzyme Q reductase, complex I dehydrogenase, DPNH-ubiquinone reductase, mitochondrial electron transport complex 1, or NADH-coenzyme Q oxidoreductase, is also shown herein to be increased in heart failure. This protein is part of the mitochondrial electron transport and oxidative phosphorylation system. Complex I is composed of about 42 different subunits. The majority of publications linking complex I with cardiomyopathies in humans and animal models report a decrease in activity (Ide T et al. Circ Res. 1999 85:357-63, Marin-Garcia J et al. J Inherit Metab Dis. 2000 23:625-33, Lucas D T et al. Am J Physiol Heart Circ Physiol 2003 284:H575-83). Increase in complex I activity has been shown in ischemia/reperfusion injury after hyperthermic stress in isolated rat hearts (Sammut I A et al. Am J Pathol. 2001 158:1821-31). Ischemia/reperfusion injury during cardiopulmonary bypass surgery in the dog induced an up-regulation of the complex I gene (Yeh C H et al. Chest. 2004 125:228-35).

Increase of complex I proteins during heart failure is believed by the inventors to be a compensatory mechanism of heart failure.

Heat shock proteins and chaperones are responsible for the correct folding and transportation of proteins and may be the first line of defense against cellular injury. Heat shock proteins and chaperones identified herein as having altered states in heart failure include 14-3-3 protein gamma, GRP 78 (78 kDa glucose-related protein), HSP 27 (heat shock protein 27), HSP 90 alpha (heat shock protein 90 alpha), protein disulfide isomerase and T-complex protein 1 theta subunit.

For example, the inventors found 14-3-3 protein gamma, also known as protein kinase C inhibitor protein 1 and KCIP-1, to be increased in heart failure. 14-3-3 protein gamma activates tyrosine and tryptophan hydroxylases in the presence of Ca(2+)/calmodulin-dependent protein kinase II, and strongly activates protein kinase C. This protein is believed to be a multifunctional regulator of the cell signaling processes mediated by both kinases. The primary function of mammalian 14-3-3 proteins is to inhibit apoptosis. 14-3-3 proteins (including gamma) are known to be involved in mammalian cell cycle control. Deregulation of 14-3-3 proteins upon overexpression of a tumor suppressor gene results in tumors in various organs of tuberous sclerosis patients. Targeted expression of dominant-negative 14-3-3 protein gamma (DN-14-3-3) to murine postnatal cardiac tissue potentiates Aski, c-jun N-terminal kinase, and p38 mitogen-activated protein kinase (MAPK) activation. DN-14-3-3 mice are unable to compensate for pressure overload, which results in increased mortality, dilated cardiomyopathy, and cardiac myocyte apoptosis due to stimulation of p38 MAPK activity (Zhang S et al. Circ Res. 2003 93:1026-8. Epub 2003 Oct. 30). Acute stress provokes lethal cardiac arrhythmias. Stress stimulates beta-adrenergic receptors, leading to cAMP elevations that can regulate HERG K+ channels both directly and via phosphorylation by cAMP-dependent protein kinase (PKA). HERG associates with 14-3-3 epsilon to potentiate cAMP/PKA effects upon HERG. The binding of 14-3-3 occurs simultaneously at the N— and C-termini of the HERG channel. 14-3-3 accelerates and enhances HERG activation, an effect that requires PKA phosphorylation of HERG and dimerization of 14-3-3. The interaction also stabilizes the lifetime of the PKA-phosphorylated state of the channel by shielding the phosphates from cellular phosphatases. The net result is a prolongation of the effect of adrenergic stimulation upon HERG activity. Thus, 14-3-3 interactions with HERG may provide a unique mechanism for plasticity in the control of membrane excitability and cardiac rhythm (Kagan A et al. EMBO J. 2002 21:1889-98).

It is the inventors' belief that an increase in 14-3-3 protein gamma may be a protective mechanism against apoptosis in heart failure.

Levels of the 78 kDa glucose-regulated protein (GRP78) were identified by the inventors to be decreased in heart failure in both human and swine. GRP78 is also known as immunoglobin heavy chain binding protein, BiP, and endoplasmic reticulum luminal Ca2+ binding protein grp78. GRP78 is a member of the heat shock protein 70 family and is believed to assist in the assembly of multimeric protein complexes inside the endoplasmic reticulum. As such, this protein is believed to be located in the lumen of the endoplasmic reticulum. Inhibition of GRP 78 expression in T-cells diminishes the anti-apoptotic effect of GRP 78 and increases activity of RNA-regulated protein kinase (PKR) in the mouse. Activated PKR catalyzes phosphorylation and inactivation of eukaryotic initiation factor (eIF) 2a. These lead to a dramatic decrease in protein synthesis inside the cell (Yang G H et al. Toxicol Appl Pharmacol. 2000 162:207-17). GRP 78 is over-expressed in the heart during organogenesis in the mouse (Barnes J A et al. Anat Embryol. 2000 202:67-74). Inhibition of GRP 78 during organogenesis leads to cardiac dysmorphogenesis. This is indicative of an important role of GRP 78 in the normal differentiation and development of the heart.

Down-regulation of GRP 78 during heart failure decreases the protection of myocytes against stress, causing increased apoptosis, reduced protein synthesis and increased misfolding of proteins within cells.

In addition, the inventors identified protein disulfide isomerase, also referred to as thyroid hormone binding protein or the beta-subunit of prolyl-4-hydroxylase, as having increased levels in heart failure. This protein catalyzes the rearrangement of —S—S-bonds in proteins via the rate-limiting reactions of oxidative formation, reduction and isomerization of disulphide bonds in the endoplasmic reticulum. This protein exhibits Ca²+-binding activity comparable to calsequestrin and calreticulin (high capacity/low affinity [Bₘₐₓ=19; 25 for calreticulin]). This protein also exhibits chaperone activity. Protein disulfide isomerase binds to triiodo-thyronine and estrogen. This binding inhibits the catalytic function of this protein. Activity of this protein is greatest in organs that synthesize disulphide-bonded proteins such as the liver, pancreas, tendon, and spleen and is lowest in brain. Endothelial cells (bovine and human) and smooth muscle cells (bovine) show increases in protein disulfide isomerase (mRNA) upon hypoxia (Graven et al. Am. J. Physiol. Lung Cell Mol. Physiol. 2002 282:L996-1003). Hypoxia and hypoxia/reoxygenation of primary astrocytes (rat) lead to up-regulation of protein disulfide isomerase (both mRNA and protein; Tanaka et al. J. Biol. Chem. 2000 275:10388-93). In vitro and in vivo over-expression of protein disulfide isomerase leads to a decrease in apoptosis after ischemia by 50% (Tanaka et al. J. Biol. Chem. 2000 275:10388-93).

The inventors believe that the increase in protein disulfide isomerase in heart failure may be a protective mechanism against decreased blood (and oxygen) supply to the enlarged heart in the later stage.

Peroxiredoxin 4, also known as Prx-IV, thioredoxin peroxidase A0372, thioredoxin-dependent peroxide reductase A0372 or antioxidant enzyme AOE372, was also found to be increased in heart failure. This protein is believed to be involved in redox regulation of the cell as well as regulation of the activation of NF-kappa-B in the cytosol by modulation of I-kappa-B-alpha phosphorylation. This protein is also an activator of c-Jun N-terminal kinase. The active site of the protein is the redox-active Cys-124 which is oxidized to Cys-SOH. Cys-SOH rapidly reacts with Cys-245-SH of the other subunit to form an intermolecular disulfide with concomitant homodimer formation. The enzyme may be subsequently regenerated by reduction of the disulfide by thioredoxin. This protein is irreversibly inactivated by overoxidation of Cys-124 (to Cys-SO3H) upon oxidative stress. The mature form of the protein is secreted from cells (Okado-Matsumoto A et al. J Biochem. 2000 127:493-501). Peroxiredoxins are the most widely represented antioxidant enzymes. In humans, Prxs are divided into two groups: isoforms 1-4, with 2 conserved motifs for N— and C-terminal cysteins and isoforms 5-6 which contain conserved Cys in the N-terminal catalytic site. The physiological contribution of the different isoforms remains undefined. A study on suppression and inhibition of Prx isoforms in human cancer cell lines by antisense constructs has shown that individual Prxs may protect against different stresses: Prxs 1-3 protect against H₂O₂ and tBHP (tert-butyl hydroperoxide), an organic oxidant; Prxs 1,2,4 protect against adriamycin (Shen C et al. Mol Med. 2002 8:95-102). A secreted and reduced form of Prx4 binds to heparin and heparin sulfate on the surface of vascular endothelial cells (Okado-Matsumoto A et al. J Biochem. 2000 127:493-501). All 6 Prxs are found in human lungs and bronchoalveolar fluid. Prx4 is the least expressed isoform in human lungs and least involved in response to pulmonary sarcoidosis (Kinnula V L et al. Thorax. 2002 57:157-64). The inventors believe that an increase in peroxiredoxin 4 indicates a response to increased oxidative stress in heart failure.

Thus, as can be seen from these experiments, while the decrease in energy and protection of cardiomyocytes against stress, apoptosis and oxygen radicals is observed with decreased levels of alpha-enolase, GRP78, ATP synthase alpha chain, NADH-ubiquinone oxidoreductase 51 kDa subunit and cytochrome C VA, the heart exhibits activation of protective mechanisms through increased levels of NADH-ubiquinone oxidoreductase 75 kDa subunit, cytochrome b5, 14-3-3 protein, protein disulfide isomerase and peroxiredoxin 4.

HSP 27, HSP 90 alpha and T-complex protein 1 theta subunit are discussed supra. HSP 27 was found to be increased in heart failure while, HSP 90 alpha was found to shift to an acidic form in heart failure. T-complex protein 1 theta subunit was found to be decreased in heart failure.

Proteases, and in particular elastase IIIB, were also identified as having an altered state in heart failure.

Elastase IIIB levels were shown by the inventors to be decreased in heart failure. This protein is also referred to as pancreatic endopeptidase E, cholesterol-binding pancreatic proteinase, pancreatic protease E, cholesterol-binding serine proteinase, and a homologue of chymotrypsin from pancreatic juice. This protein is a member of the trypsin family of serine proteases and acts as an efficient protease with alanine specificity but only little elastolytic activity.

Miscellaneous other proteins were also identified herein as having an altered state in heart failure.

For example, annexin V levels were shown by the inventors to decrease during heart failure. Annexin V, also known as lipocortin V, endonexin II, calphobindin I, CBP-I, placental anticoagulant protein I, PAP-I, PP4, thromboplastin inhibitor, vascular anticoagulant-alpha, VAC-alpha, and anchorin CII, is a potent anticoagulant and inhibitor of prothrombin activation. This protein also inhibits phospholipase A2 activity and protein kinase C (PKC). Further, annexin V forms voltage-gated Ca²⁺ channels and is involved in regulation of cell differentiation in response to growth factors, maintenance of cytoskeletal organization and regulation of membrane interaction during exocytosis. Annexin V is believed to bind SERCA2 and increases SR Ca²⁺-ATPase activity. It is partly relocated from the cell to the interstitium in end-stage heart failure in humans (Benevolensky D et al. Lab Invest. 2000 80:123-33). The down-regulation of annexin V is believed to lead to a decrease in PKC inhibition and an enhancement of PKC mediated membrane-associated processes involving phosphatidylserine and diacylglycerol.

The decreased activation of SERCA2 is expected by the inventors to lower the Ca²⁺ reuptake to the sarcoplasmic reticulum and increase intracellular [Ca²⁺] during diastole. Both of these effects are believed to be enhanced by the relocation of annexin V to the interstitium.

Levels of beta-lactoglobulin 1A and 1C were also identified by the inventors as being increased during heart failure. This protein is the primary component of whey. The protein binds retinol and is believed to be involved in the transport of retinol. Glycodelin or PP14 protein is the human equivalent of beta-lactoglobulin. Beta-lactoglobulin 1A and 1C are members of the lipocalin family. Lipocalins are a diverse, interesting, yet poorly understood family of proteins composed, in the main, of extracellular ligand-binding proteins displaying high specificity for small hydrophobic molecules. Functions of these proteins include transport of nutrients, control of cell regulation, pheromone transport, cryptic coloration and the enzymatic synthesis of prostaglandins. Glycodelin or PP14 protein, the human equivalent of this protein, is secreted into the endometrium from mid-luteal phase of the menstrual cycle and during the first semester of pregnancy.

Chloride intracellular channel protein 1, also referred to as CLI1, nuclear chloride ion channel 27, NCC27, p64 CLCP or chloride channel ABP, stabilizes membrane potential and controls muscle cell excitability and is increased in heart failure. CLI1 mRNA is upregulated during slow-to-fast fiber type transition during unloading induced muscle disuse in the rat. This is accompanied by an increase in resting membrane chloride conductance without changes in MHC type and is seen as an early adaptation to modified use (Pierno S et al. Brain. 2000 125:1510-21). The slow-to-fast transition is also accompanied by the upregulation of SR Ca²⁺ ATPase.

The inventors believe that a switch in excitation-contraction characteristics of cardiomyocytes towards those of a fast twitch fiber during heart failure may allow for an increase in heart rate due to faster re-polarization. This may serve as a compensatory mechanism in the early stages of heart failure development to sustain cardiac output. Long term, however, it increases energy demand and fatigability of the heart.

The inventors also found proliferating cell nuclear antigen, also known as PCNA and cyclin, to be increased in heart failure. This protein is an auxiliary protein of DNA polymerase delta and is involved in the control of eukaryotic DNA replication by increasing the polymerase's processibility during elongation of the leading strand. PCNA is expressed in atherosclerotic carotid plaques (Lavezzi A M et al. Int J Cardiol. 2003 92:59-63), myocarditis (Arbustine E et al. Am J Cardiol. 1993 72:608-14), ventricular hypertrophy induced by renovascular hypertension (Buzello M et al. Virchows Arch. 2003 442:364-71. Epub 2003 Apr. 02) and during embryogenesis and early postnatal life which is characterized by cardiomyocyte hyperplasia (Petrovic D et al. Cardiovasc Pathol. 2000 9:149-52). PCNA index is highest in human hypertrophic cardiomyopathy compared to post-MI remodeling and hypertension due to elevated number of hyperdiploid cells. The number of apoptotic cells is initially low, but rises at the end of the hypertrophic process in order to eliminate the hyperdiploid cardiomyocytes (Matturri L et al. Int J Cardiol. 2002 82:33-39). It has also been found in sera from patients with systemic lupus erythematosus and malignant lymphoma (Takasaki Y et al. J Immunol. 2001 166:4780-7).

As will be understood by one of skill in the art upon reading this patent application, additional proteins involved in cell organization, metabolic proteins, heat shock proteins and chaperones, and proteases, as well miscellaneous proteins to those exemplified herein may exhibit altered states in heart failure and can be used in accordance with the teachings of the present invention, alone or in profiles with these proteins exemplified herein to diagnose, stage, monitor and treat heart failure.

Collectively these data are indicative of alterations in the proteome occurring in the failing heart. Further, these data are indicative of an altered protein state or a profile of proteins in altered states being useful in diagnosing, monitoring, staging and treating heart failure, as well as in identifying and monitoring treatments for heart failure. It is expected that levels of proteins in the profiles may change with the stage of disease in a subject (e.g., pre-symptomatic heart failure, early, mid or late stage heart failure; New York Heart Association Functional Classes I, II, III, IV; or ACC/AHA Stages of Heart Failure A, B, C and D). Some proteins may be general markers of heart failure and found at several or all stages of the disease. Other proteins or protein profiles may be stage specific and found only at a particular stage of the disease.

For example, the inventors have found that in the swine model levels of proteins 2-oxoisovalerate dehydrogenase beta, beta-lactoglobulin 1A and 1C and chloride intracellular channel protein 1 were elevated at 2 weeks and 6 weeks and levels of proteins NADH ubiquinone oxidoreductase 30 kDA and peroxiredoxin 4 were elevated at 2 weeks and 4 weeks.

In one embodiment, a protein profile is generated comprising altered states of two or more metabolic proteins, heat shock proteins, proteins involved in cellular organization, proteases and/or miscellaneous proteins. In a preferred embodiment, the two or more proteins of the profile are selected from different functional groups, e.g., one being a metabolic protein and the other being a heat shock protein or one being a protein involved in cellular organization and the other being a miscellaneous protein, etc. More preferred is a profile comprising at least one protein from each functional group.

In this embodiment, it is preferred that the two or more proteins be selected from 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin. Most preferred is a profile comprising all of these proteins.

Multiple proteins of this profile with altered states have not been disclosed in other proteomic studies of heart failure. In fact, this is the first association for some of these alterations with heart failure. Thus, in another embodiment, an altered state of one or more proteins selected from 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3 and T-complex protein 1 theta subunit, for which no association with heart failure had been previously established, can be measured to diagnose, stage, monitor and treat heart failure.

The unique protein profiles generated according to the present invention are useful in the diagnosis of heart failure in a subject. In this embodiment, a protein profile is generated from a biological sample obtained from a subject suspected of suffering from heart failure. Examples of biological samples that can be used for generating a profile of the present invention include, but are not limited to, tissue samples, whole blood, blood cells, serum, plasma, cytolyzed blood (e.g., by treatment with hypotonic buffer or detergents; see, e.g., International Patent Publication No. WO 92/08981, published May 29, 1992), urine, cerebrospinal fluid, and lymph. This protein profile is then compared to a profile of the same proteins in a healthy control. A protein profile wherein 14-3-3 protein gamma, beta lactoglobulin 1A and 1C, 2-oxoisovalerate dehydrogenase beta, chloride intracellular channel protein 1, cytochrome b5, F-actin capping protein beta 1, glycogen phosphorylase, HSP 27, hUNC-45, MTCBP-1, long chain fatty acid CoA ligase 1, 6-phosphofructokinase, NADH-ubiquinone oxidoreductase 30 kDa subunit, NADH-ubiquinone oxidoreductase 75 kDa subunit, peroxiredoxin 4, proliferating cell nuclear antigen, protein disulphide isomerase, TnI and/or TnC is elevated and/or alpha-actinin, alpha-enolase, annexin V, aspartate aminotransferase, ATP synthase alpha chain, cytochrome C oxidase VA, desmin, dihydrolipoamide dehydrogenase, elastase IIIB, fructose-bisphosphate aldolase A, fumarate hydratase, GRP78, moesin, ventricular MLC1, MLC2, NADH ubiquinone oxidoreductase 51 kDa subunit, stathmin 3, T-complex protein 1 theta subunit, tropomyosin alpha 1, tropomyosin beta, troponin T, vimentin and/or the degraded form of MRP 1 is decreased and/or tubulin alpha and/or tubulin beta are shifted towards the myofilament fraction of the myocytes and/or novel enoyl CoA isomerase is shifted to its acidic form and/or the ratio between metavinculin and vinculin is shifted towards metavinculin and/or HSP 90 alpha is shifted to its acidic form, as compared to the same proteins in the healthy control, is indicative of heart failure.

The comparisons between levels or states of proteins performed according to the invention may be straight-forward comparisons, such as a ratios, or may involve weighting of one or more of the measures relative to, for example, their importance to the particular situation under consideration. Comparison may also involve subjecting the measurement data to any appropriate statistical analysis. This applies not only to comparison of metavinculin and vinculin, but to comparison of any two or more proteins of interest according to the invention.

Protein profiles generated according to the present invention are also useful in monitoring the condition of a subject with heart failure. In this embodiment, states of proteins are monitored in the subject over a selected time period. Monitoring periods can be selected routinely by those of skill in the art based upon the severity of the condition being monitored. For example, yearly monitoring may begin at age 50 in all subjects, particularly those with any family history of cardiovascular disease. For subjects at high risk for myocardial infarction and/or heart failure, monitoring may be performed on an outpatient basis quarterly, bimonthly or even monthly. Subjects with stable or unstable angina may also be monitored at similar time points on an outpatient basis. Subjects diagnosed with myocardial infarction or with a history of heart failure may be monitored more frequently. Proteins monitored may include one or more of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin. Proteins selected for monitoring may be from the same functional protein group or from different functional protein groups. By functional protein group, as used herein, it is meant proteins involved in cell organization, metabolic proteins, heat shock proteins, proteases as well as miscellaneous proteins. More preferred is monitoring of two or more of these proteins selected from different functional protein groups. Most preferred is monitoring of most or all of these proteins. A protein profile wherein 14-3-3 protein gamma, beta lactoglobulin 1A and 1C, 2-oxoisovalerate dehydrogenase beta, chloride intracellular channel protein 1, cytochrome b5, F-actin capping protein beta 1, glycogen phosphorylase, HSP 27, hUNC-45, MTCBP-1, long chain fatty acid CoA ligase 1, 6-phosphofructokinase, NADH-ubiquinone oxidoreductase 30 kDa subunit, NADH-ubiquinone oxidoreductase 75 kDa subunit, peroxiredoxin 4, proliferating cell nuclear antigen, protein disulphide isomerase, TnI and/or TnC is elevated and/or alpha-actinin, alpha-enolase, annexin V, aspartate aminotransferase, ATP synthase alpha chain, cytochrome C oxidase VA, desmin, dihydrolipoamide dehydrogenase, elastase IIIB, fructose-bisphosphate aldolase A, fumarate hydratase, GRP78, moesin, ventricular MLC1, MLC2, NADH ubiquinone oxidoreductase 51 kDa subunit, stathmin 3, T-complex protein 1 theta subunit, tropomyosin alpha 1, tropomyosin beta, troponin T, vimentin and/or the degraded form of MRP 1 is decreased and/or tubulin alpha and/or tubulin beta are shifted towards the myofilament fraction of the myocytes and/or novel enoyl CoA isomerase is shifted to its acidic form and/or the ratio between metavinculin and vinculin is shifted towards metavinculin and/or HSP 90 alpha is shifted to its acidic form, as compared to the same proteins in a healthy control or the same subject at a time period prior to generation of this protein profile, is indicative of the subject approaching or having heart failure.

Monitoring of the protein profile of the present invention can also be used in the selection of different treatment regimes for subjects suffering from severe heart failure versus less severe heart failure. For example, more aggressive treatment regimes may be selected for subjects suffering from severe heart failure while less aggressive treatment regimes may be selected for those subjects suffering from less severe heart failure heart failure.

It is believed that the identified proteins and protein profiles generated in accordance with the present invention can be also be used to stage progression of heart failure in a subject. In this method, diseased controls comprising protein profiles of the present invention are established for various stages of heart failure ranging from initial insult to end-stage. A protein state or protein profile for a subject can then be generated and compared to the protein states or protein profiles of the diseased controls to determine what stage of progression of heart failure the subject is at.

Another aspect of the present invention relates to a method for evaluating treatment of a subject with heart failure. As discussed, supra, a protein profile wherein 14-3-3 protein gamma, beta lactoglobulin 1A and 1C, 2-oxoisovalerate dehydrogenase beta, chloride intracellular channel protein 1, cytochrome b5, F-actin capping protein beta 1, glycogen phosphorylase, HSP 27, hUNC-45, MTCBP-1, long chain fatty acid CoA ligase 1, 6-phosphofructokinase, NADH-ubiquinone oxidoreductase 30 kDa subunit, NADH-ubiquinone oxidoreductase 75 kDa subunit, peroxiredoxin 4, proliferating cell nuclear antigen, protein disulphide isomerase, TnI and/or TnC is elevated and/or alpha-actinin, alpha-enolase, annexin V, aspartate aminotransferase, ATP synthase alpha chain, cytochrome C oxidase VA, desmin, dihydrolipoamide dehydrogenase, elastase IIIB, fructose-bisphosphate aldolase A, fumarate hydratase, GRP78, moesin, ventricular MLC1, MLC2, NADH-ubiquinone oxidoreductase 51 kDa subunit, stathmin 3, T-complex protein 1 theta subunit, tropomyosin alpha 1, tropomyosin beta, troponin T, vimentin and/or the degraded form of MRP 1 is decreased and/or tubulin alpha and/or tubulin beta are shifted towards the myofilament fraction of the myocytes and/or novel enoyl CoA isomerase is shifted to its acidic form and/or the ratio between metavinculin and vinculin is shifted towards metavinculin and/or HSP 90 alpha is shifted to its acidic form, as compared to the same protein or proteins in a healthy control or the same subject at a time period prior to generation of this protein profile, is indicative of the subject having heart failure, and more particularly progressive heart failure. Accordingly, a subject can be administered a known treatment or a potential new treatment and his/her protein profile evaluated to assess whether the treatment or potential new treatment alters the protein profile in a manner indicative of the subject improving.

For example, a protein profile indicative of the subject improving may comprise a protein profile wherein 14-3-3 protein gamma, beta lactoglobulin 1A and 1C, 2-oxoisovalerate dehydrogenase beta, chloride intracellular channel protein 1, cytochrome b5, F-actin capping protein beta 1, glycogen phosphorylase, HSP 27, hUNC-45, MTCBP-1, long chain fatty acid CoA ligase 1, 6-phosphofructokinase, NADH-ubiquinone oxidoreductase 30 kDa subunit, NADH-ubiquinone oxidoreductase 75 kDa subunit, peroxiredoxin 4, proliferating cell nuclear antigen, protein disulphide isomerase, TnI and/or TnC is decreased and/or alpha-actinin, alpha-enolase, annexin V, aspartate aminotransferase, ATP synthase alpha chain, cytochrome C oxidase VA, desmin, dihydrolipoamide dehydrogenase, elastase IIIB, fructose-bisphosphate aldolase A, fumarate hydratase, GRP78, moesin, ventricular MLC1, MLC2, NADH ubiquinone oxidoreductase 51 kDa subunit, stathmin 3, T-complex protein 1 theta subunit, tropomyosin alpha 1, tropomyosin beta, troponin T, vimentin and/or the degraded form of MRP 1 is increased and/or tubulin alpha and/or tubulin beta are not shifted towards the myofilament fraction of the myocytes and/or novel enoyl CoA isomerase is not shifted to its acidic form and/or the ratio between metavinculin and vinculin is not shifted towards metavinculin and/or HSP 90 alpha is not shifted to its acidic form, as compared to the same protein or proteins in the same subject at a time period prior to administration of the known or potential new treatment.

However, as will be understood by those of skill in the art upon reading this disclosure, some altered protein states observed herein may be compensatory and/or desirable and thus the opposite effect to the state of the protein to that observed in heart failure may not always be required. In a preferred embodiment, the protein profile monitored to assess the known or potential new treatment comprises a profile wherein two or more of the proteins are selected from different functional groups of proteins, namely proteins involved in cellular organization, metabolic proteins, heat shock proteins, protease as well as miscellaneous proteins. Most preferred is use of a protein profile comprising all or most of the proteins 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin to assess the known or potential new treatment.

The present invention also provides methods for treating a subject with heart failure. In this aspect of the present invention, a subject is administered a therapeutic agent which alters the state of one or more proteins of the heart failure protein profile. For example, a therapeutic agent of the invention may alter the state of one or more of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin. A preferred agent may be one which alters the protein state by decreasing the level of 14-3-3 protein gamma, beta lactoglobulin 1A and 1C, 2-oxoisovalerate dehydrogenase beta, chloride intracellular channel protein 1, cytochrome b5, F-actin capping protein beta 1, glycogen phosphorylase, HSP 27, hUNC-45, MTCBP-1, long chain fatty acid CoA ligase 1, 6-phosphofructokinase, NADH-ubiquinone oxidoreductase 30 kDa subunit, NADH-ubiquinone oxidoreductase 75 kDa subunit, peroxiredoxin 4, proliferating cell nuclear antigen, protein disulphide isomerase, TnI and/or TnC and/or increasing the level of alpha-actinin, alpha-enolase, annexin V, aspartate aminotransferase, ATP synthase alpha chain, cytochrome C oxidase VA, desmin, dihydrolipoamide dehydrogenase, elastase IIIB, fructose-bisphosphate aldolase A, fumarate hydratase, GRP78, moesin, ventricular MLC1, MLC2, NADH ubiquinone oxidoreductase 51 kDa subunit, stathmin 3, T-complex protein 1 theta subunit, tropomyosin alpha 1, tropomyosin beta, troponin T, vimentin and/or the degraded form of MRP 1 and/or inhibiting the shift of tubulin alpha and/or tubulin beta towards the myofilament fraction of the myocytes and/or the shift of novel enoyl CoA isomerase to its acidic form and/or the shift of the ratio between metavinculin and vinculin towards metavinculin and/or the shift of HSP 90 alpha to its acidic form. However, as will be understood by those of skill in the art upon reading this disclosure, some altered protein states observed herein may be compensatory and/or desirable and thus the opposite effect to the state of the protein to that observed in heart failure may not always be required for a useful therapeutic agent. Most preferred are therapeutic agents that alter the state of more than one protein of this profile.

Also provided in the present invention are methods for screening for agents potentially useful in modulating heart failure by assessing the ability of an agent to modulate a state of one or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin. In this screening assay, the ability of an agent to modulate the state of at least one and more preferably two or more of these proteins is indicative of the agent being a modulator of heart failure. Such assays can be performed routinely by those of skill in the art based upon well-known techniques for high throughput screening assays. In a preferred embodiment, such assays are performed in cell culture, for example cardiac cells and a change or modulation of the state of one ore more proteins is assessed in the presence and absence of the agent. Agents which modulate at least one and more preferably two or more of these proteins may be useful in the treatment of heart failure.

The present invention also provides methods for identifying agents that modulate progression of heart failure. In these methods, protein profiles of the present invention are obtained in the swine model for ischemic heart failure in the presence and absence of a test agent and compared to corresponding proteins profiles for appropriate controls. A change in the protein profile upon administration of the test agent as compared to the control protein profile is indicative of the test agent being a modulator of heart failure. In one embodiment of this method, a protein profile of the present invention is detected in a LAD-ligated swine. The swine is then administered a test agent and the corresponding profile of proteins is detected again in the swine. A change in the corresponding protein profile detected after administration of the test agent as compared to the protein profile detected prior to administration of the test agent is indicative of the test agent being a modulator of heart failure. However, as will be understood by the skilled artisan upon reading this patent application, alternative comparisons and controls can be made. For example, the control may comprise a corresponding profile in a different pig or pigs at a selected stage of heart failure.

By “modulator” it is meant to include agents that improve or inhibit the progression of heart failure as well as agents which worsen the progression of heart failure. Examples of such agents include, but are in no way limited to small organic molecules, proteins, peptides, peptidomimetics, antisense molecules, and ribozymes. Agents which improve or inhibit the progression of heart failure in the LAD-ligated swine are expected to be useful in the treatment of heart failure in other mammals, including humans, and may be referred to herein as therapeutic agents. Such therapeutic agents may act by decreasing the level of 14-3-3 protein gamma, beta lactoglobulin 1A and 1C, 2-oxoisovalerate dehydrogenase beta, chloride intracellular channel protein 1, cytochrome b5, F-actin capping protein beta 1, glycogen phosphorylase, HSP 27, hUNC-45, MTCBP-1, long chain fatty acid CoA ligase 1, 6-phosphofructokinase, NADH-ubiquinone oxidoreductase 30 kDa subunit, NADH-ubiquinone oxidoreductase 75 kDa subunit, peroxiredoxin 4, proliferating cell nuclear antigen, protein disulphide isomerase, TnI and/or TnC and/or increasing the level of alpha-actinin, alpha-enolase, annexin V, aspartate aminotransferase, ATP synthase alpha chain, cytochrome C oxidase VA, desmin, dihydrolipoamide dehydrogenase, elastase IIIB, fructose-bisphosphate aldolase A, fumarate hydratase, GRP78, moesin, ventricular MLC1, MLC2, NADH ubiquinone oxidoreductase 51 kDa subunit, stathmin 3, T-complex protein 1 theta subunit, tropomyosin alpha 1, tropomyosin beta, troponin T, vimentin and/or the degraded form of MRP 1 and/or inhibiting the shift of tubulin alpha and/or tubulin beta towards the myofilament fraction of the myocytes and/or the shift of novel enoyl CoA isomerase to its acidic form and/or the shift of the ratio between metavinculin and vinculin towards metavinculin and/or the shift of HSP 90 alpha to its acidic form in the LAD-ligated swine. However, as will be understood by those of skill in the art upon reading this disclosure, some altered protein states observed herein may be compensatory and/or desirable and thus the opposite effect to the state of the protein to that observed in heart failure may not always be required for a useful therapeutic agent. Most preferred are agents that alter the state of more than one protein of this profile. Agents which alter the protein state by increasing the level of 14-3-3 protein gamma, beta lactoglobulin 1A and 1C, 2-oxoisovalerate dehydrogenase beta, chloride intracellular channel protein 1, cytochrome b5, F-actin capping protein beta 1, glycogen phosphorylase, HSP 27, hUNC-45, MTCBP-1, long chain fatty acid CoA ligase 1, 6-phosphofructokinase, NADH-ubiquinone oxidoreductase 30 kDa subunit, NADH-ubiquinone oxidoreductase 75 kDa subunit, peroxiredoxin 4, proliferating cell nuclear antigen, protein disulphide isomerase, TnI and/or TnC and/or decreasing the level of alpha-actinin, alpha-enolase, annexin V, aspartate aminotransferase, ATP synthase alpha chain, cytochrome C oxidase VA, desmin, dihydrolipoamide dehydrogenase, elastase IIIB, fructose-bisphosphate aldolase A, fumarate hydratase, GRP78, moesin, ventricular MLC1, MLC2, NADH ubiquinone oxidoreductase 51 kDa subunit, stathmin 3, T-complex protein 1 theta subunit, tropomyosin alpha 1, tropomyosin beta, troponin T, vimentin and/or the degraded form of MRP 1 and/or inducing the shift of tubulin alpha and/or tubulin beta towards the myofilament fraction of the myocytes and/or the shift of novel enoyl CoA isomerase to its acidic form and/or the shift of the ratio between metavinculin and vinculin towards metavinculin and/or the shift of HSP 90 alpha to its acidic form, in the LAD-ligated swine are potentially detrimental to the heart and may have serious side effects, particularly in subjects with heart failure. However, as will be understood by those of skill in the art upon reading this disclosure, some altered protein states observed herein may be compensatory and/or desirable.

The methods of the invention can be performed at the point of care by appropriately trained personnel. For example, emergency medical service workers can perform a diagnosis of the invention at the site of a medical emergency or in an ambulance on the way to the hospital. Similarly, medical personal in an emergency room, cardiac care facility or other point of care location at a hospital can perform diagnosing, monitoring, staging, evaluating treatments, and treating heart failure according to the invention themselves. Naturally and where clinically appropriate, the patient biological sample, such as blood or any blood product, plasma, or serum, or urine, or cerebrospinal fluid, or lymph, may be provided to a hospital laboratory to perform the test.

The invention extends to test materials including reagents in a kit form for the practice of the inventive methods. The materials may comprise binding partners that are specific to the proteins under detection, and in one embodiment, comprise an antibody or antibodies, each of which is specific for one of each of the proteins, the presence of which is to be determined. “Specific” as used herein refers to the specificity of a binding partner, e.g., an antibody for a protein, i.e., there is no, or minimal, cross reaction of the binding partner with other proteins or materials in the sample under test. The protein(s) can be either in the native or mature form or can be detectable, e.g., immunologically detectable, modified forms of the protein, including fragments thereof that are immunologically detectable. By “immunologically detectable” is meant that the protein and/or modified forms thereof contain an epitope that is specifically recognized by a given antibody.

In an illustrative embodiment, one antibody of each pair specific for a particular protein is irreversibly immobilized onto a solid support; this antibody is alternately referred to hereinafter as a capture antibody. The other antibody specific for the same protein is labeled, and is capable of moving with a sample to the location on the solid support of the capture antibody. This antibody is sometimes referred to herein as the detection antibody.

Binding of the binding partner, e.g., antibody, to its antigen, the protein, in a sample can be detected by any suitable detection means, such as optical detection, biosensors, homogenous immunoassay formats, and the like. For example, particular optical sensing systems and corresponding devices are contemplated and are discussed in U.S. Pat. No. 5,290,678.

As used herein, the term antibody includes polyclonal and monoclonal antibodies of any isotype (IgA, IgG, IgE, IgD, IgM), or an antigen-binding portion thereof, including but not limited to F(ab) and Fv fragments, single chain antibodies, chimeric antibodies, humanized antibodies, and a Fab expression library.

Antibodies useful as detector and capture antibodies in the present invention may be prepared by standard techniques well known in the art. The antibodies can be used in any type of immunoassay. This includes, for example, two-site sandwich assays and single site immunoassays of the non-competitive type, as well as traditional competitive binding assays.

For example, the sandwich or double antibody assay, of which a number of variations exist, provides ease and simplicity of detection, and the ability to quantify the protein detected. In a typical sandwich assay, unlabeled antibody is immobilized on a solid phase, e.g. microtiter plate, and the sample to be tested is added. After a certain period of incubation to allow formation of an antibody-antigen complex, a second antibody, labeled with a reporter molecule capable of inducing a detectable signal, is added and incubation is continued to allow sufficient time for binding with the antigen at a different site, resulting with a formation of a complex of antibody-antigen-labeled antibody. The presence of the antigen is determined by observation of a signal which may be quantitated by comparison with control samples containing known amounts of antigen.

The assays may be, for example, competitive assays, sandwich assays, and the label may be selected from the group of well-known labels such as, for example, radioimmunoassay, fluorescent or chemiluminescence immunoassay, or immunoPCR technology. Extensive discussion of such known immunoassay techniques is not required here since these are known to those of skill in the art. See, for example, Takahashi et al. (Clin Chem 1999 45:1307) for S100B assay.

The contents of all references, pending patent applications, and published patents cited throughout this application are hereby expressly incorporated by reference.

The following nonlimiting examples are provided to further illustrate the present invention.

### EXAMPLES

### Example 1

### Protein Extraction Procedure for Human Heart Tissue

Human left ventricular tissue was obtained, snap frozen after explantation and stored at −80°. For analysis, approximately 75 kg of tissue was homogenized at 4° C. in extraction buffer containing 20 mM Tris pH 6.8, 7 M urea, 2 M thiourea, 4% amidosulfobetaine-14 containing a proteinase, kinase and phosphatase inhibitor cocktail (0.2 mM sodium vanidate, 50 mM sodium fluoride, 2 mM EDTA, 1 μM leupeptin, 1 μM pepstatin A, 0.36 μM aprotinin, 0.25 mM phenylmethyl-sulfonyl fluoride) at the ratio of tissue to buffer 1:10 w/v.

### Example 2

### 2-Dimensional Electrophoresis of Proteins Extracted from Human Heart Tissue

Equal protein loads were determined based on Commassie stained 1D SDS-PAGE gels. Tissue homogenate was applied onto Immobilized pH gradient (IPG) ReadyStrips pH 3-10 (17 cm, Bio-Rad, Hercules, Calif., USA). IEF was carried out according to the manufacturer's protocol (Protean IEF cell (Bio-Rad)). IPG strips were actively rehydrated at 50 V for 10 hours, then a rapid voltage ramping method was applied as follows: 100 V for 25 Vh, 500 V for 125 Vh, 1000 V for 250 Vh, and the isoelectrically focused to accumulate 65 kVh using focusing buffer containing 2 mM EDTA, 62.5 mM DTT, 8 M urea, 2.5 M thiourea and 4% CHAPS.

The second dimension was run by a the method of Laemmli (Nature 1970 227:680-685) (with 0.125% SDS compared to 0.1%) on a 8% resolving, 4.5% stacking polyacrylamide gel at 50 V (overnight) followed by silver staining according to Shevchenko et al. (Anal. Chem. 1996, 68,850-858).

### Example 3

### Ischemia Induced Failing Heart Model in Swine

Neutered male swine (13-34.0 kg) underwent open chest surgery for occlusion of the mid-third of the left anterior descending branch of coronary artery (LAD). Sham-operated swine (SHAM) under went the same surgical procedure except the LAD was not occluded. During open chest surgery and at termination, animals were under general anesthesia (a preanaesthetic, atropine followed by a combination of ketamine, midazolam and isoflurane, with anesthesia maintained by isoflurane). Upon recovery the animals received analgesics as needed. At 4 weeks, echocardiography was performed on conscious mildly sedated animals. To estimate the left ventricle ejection fraction echocardiographs were performed in the lateral position, left side of the swine down, using a PieMedical 200 scanner equipped with a 5.0/7.5 mHz probe. At 6 weeks post-surgery animals were sacrificed (n=9 LAD, n=5 SHAM), the hearts were excised, left ventricle sectioned on infarcted, intermediate and remote from infarction areas and immediately snap-frozen in liquid nitrogen, stored at −80° C. To assess the development of heart failure LAD ligated animals as well as corresponding SHAMs were terminated at 2 (n=4 LAD, n=3 SHAM) and 4 (n=4 LAD, n=4 SHAM) weeks post-surgery and tissue and blood samples were collected in the same manner as above. To investigate protein changes at the initial time point of ischemic injury, animals were terminated after 30 minutes of LAD occlusion. Each group of experimental animals (n=6 LAD, n=3 SHAM). All experimental procedures conformed to guidelines of the Canadian Council of Animal care and were approved by Queen's University Animal Care Committee.

### Example 4

### Protein Extraction Procedure for Swine Heart Tissue

For analysis, swine left ventricular tissue was obtained from an area remote from infarction site. Approximately 0.2-0.3 mg of tissue was homogenized at 4° C. in extraction buffer containing 50 mM Tris pH 6.8, 100 mM sodium chloride, 1% SDS, 10% glycerol containing a proteinase, kinase and phosphatase inhibitor cocktail (0.2 mM sodium vanidate, 50 mM sodium fluoride, 2 mM EDTA, 1 μM leupeptin, 1 W pepstatin A, 0.36 μM aprotinin, 0.25 mM phenylmethyl-sulfonyl fluoride). The suspension was centrifuged at 16000×g for 5 min, supernatant collected and stored frozen at −80° C. Total protein concentration in each sample was determined by Lowry assay prior the experiments.

### Example 5

### “IN Sequence”—Subproteome Extraction for Swine Heart Tissue

Heart tissue from a non-infarcted region of the left ventricle was homogenized in 20 mM Tris (pH 6.8) and 0.2 mM sodium vanidate, 50 mM sodium fluoride, 2 mM EDTA, 1 μM leupeptin, 1 μM pepstatin A, 0.36 μM aprotinin, 0.25 mM phenylmethylsulfonyl fluoride at 4° C. at the ratio of tissue weight:buffer volume=1:4 (whole tissue homogenate). The homogenate was centrifuged at 4° C. for 5 minutes at 16000×g (as are all centrifugation), the supernatant removed and the pellet re-extracted with the same buffer. Both supernatants were combined as cytoplasmic extract (extract #1). The remaining pellet was extracted by homogenization in 0.05% aqueous trifluoroacetic acid (TFA) (v/v) and 1 mM TCEP Tris(2-carboxyethyl)phosphine hydrochloride) (initial tissue weight:buffer volume=1:4) at 4° C. The sample was centrifuged and the pellet re-extracted with the same buffer. Both supernatants were combined as myofilament enriched extract (extract #2). Extracts were stored at −80° C. Total protein concentration in each sample was determined by Bradford assay (Bio-Rad Protein Assay Reagent, Bio-Rad, Hercules, Calif., USA).

### Example 6

### 2-DE Protein Separation for Swine Heart Tissue

Protein separation was carried out in accordance with procedures described by Neverova and Van Eyk (Proteom. 2000 2:22-31) and Arrell et al. (Circ. Res. 2001 88:763-773). Proteins were resolved by 2-DE gel electrophoresis (2-DE). Immobilized pH gradient (IPG) Ready Strips (170 mm, BioRad) with a linear pH range of 3-10, 4-7 or 4.7-5.9 were used to separate proteins in the first dimension, by isoelectric focusing (IEF). 200 or 750pg of total protein was dissolved in rehydration buffer containing 8 mol/L urea, 2.5 mol/L thiourea, 0.5% ampholytes (pH 3.5-10, Sigma), 4% 3-[(3-cholamidopropyl)dimethyammonio]-1-propane-sulfonate (CHAPS), 2 mmol/L ethylenediaminetetraacetic acid EDTA and 100 mmol/L dithiothreitol (DTT) prior to application to IPG strips. IPG strips were actively rehydrated at 50V for 10 hours with 500 μl of prepared protein sample. IPG strips were then subjected to 100V for 25 volt-hours, 500V for 125 volt-hours, 1000V for 250 volt-hours and 8000V for 65000 volt-hours at a temperature maintained at 20° C. Upon completion of IEF, IPG strips were stored at −20° C.

Prior to the second dimension SDS-PAGE protein separation IPG strips were equilibrated in a buffer containing 50 mmol/L Tris-Cl, pH 8.8, 6 mol/L urea, 30% glycerol (v/v), 2% SDS (w/v) and 64 mmol/L DTT for 20 minutes and then incubated in a similar buffer containing 0.14 mol/L iodoacetamide instead of 64 mmol/L DTT, for 20 minutes. Equilibrated IPG strips were applied to 4.5% stacking/12.5% resolving SDS-PAGE gels using a Protean II XL system (BioRad) and SDS-PAGE was conducted at 100V for 30 minutes followed by 250V for 4.5 hours.

### Example 7

### Protein Visualization on 2-D gels of Swine and Human Heart Tissue

Protein visualization was carried out in accordance with procedures described by Neverova and Van Eyk (Proteom. 2000 2:22-31) and Arrell et al. (Circ. Res. 2001 88:763-773). Subsequent to electrophoresis, 2-DE gels were fixed in a solution containing 50% methanol/10% acetic acid and protein spots were visualized by silver staining according to a previously described method (Shevchenko et all. Anal. Chem. 1996, 68,850-858), which is compatible with mass spectrometry.

### Example 8

### Selection of Candidate Protein Spots for Identification (Human and Swine)

A number of criteria were used for appropriate selection of protein spots for identification. These criteria were employed in order to ensure that (i) the protein spot alteration was relevant to a large proportion of the sample population (ii) there was a sufficient degree of resolution of the protein spot of interest on the 2-DE gel to allow excision of only the spot of interest and (iii) the degree of resolution of the protein spot of interest on the 2-DE gel permitted confidence in its alignment with the same spot on multiple 2-DE gel images. Lastly, the protein spot of interest was required to be relatively isolated in relation to other protein spots in the vicinity and be relatively easily identifiable on distinct 2-DE gels. In the case of low abundance proteins, it was necessary to excise a given protein spot from multiple 2-DE gels (up to ten 2-DE gels) in order to obtain sufficient quantities for mass spectrometric analysis. However, in some cases, we were still unable to isolate sufficient quantities of protein to allow for identification.

### Example 9

### Image Analysis of 2-D gels of Human and Swine Heart Tissue

Two-dimensional gel image analysis was carried out in accordance with procedures described by Neverova and Van Eyk (Proteom. 2000 2:22-31) and Arrell et al. (Circ. Res. 2001 88:763-773). Two-dimensional gel images were acquired, at a resolution of 150 dpi, using a PowerLook II scanner (UMAX Data Systems Inc.) on a Sun Ultra 5 computer (Sun Microsystems Inc.). Protein spots were detected, quantified and matched from multiple 2-DE gel images, for the creation of composite images, using Investigator HT Proteome Analyzer 1.0.1 software (Genomic Solutions). Two composite images were generated for each subproteome, each representing a normalized average of five 2-DE gel images derived from analysis of either normal or diseased tissue samples. The method of match-spot normalization was utilized to compensate for gel to gel variation. For human study a two tailed unequal variance student T-test analysis was employed to determine statistically significant differences in mean integrated intensities of corresponding protein spots from normal and ICM composite images. A difference with an associated p-value of less than 0.05 was considered statistically significant.

### Example 10

### Protein Identification by MS

Protein spots excised from silver-stained 2D gels were destained according to Gharahdaghi et al. (Electrophoresis 1999, 20, 601-605). Briefly, gel pieces were incubated for 10 minutes in 50 mM sodium thiosulfate followed by 15 mM potassium ferricyanide solubilize silver. The destaining solution was removed, and the gels washed with 3×10 minutes in water, until the yellow colour disappeared, then incubated with 100% acetonitrile for 5 minutes and finally dried under vacuum before enzymatic digestion with sequence-grade modified trypsin (Promega, Madison, Wis.) as previously described Arrell et al. (Circ. Res. 2001 88:763-773). Tryptic peptides were extracted with 50% ACN/5% TFA, dried under vacuum, and reconstituted with 3 μL of 50% ACN/0.1% TFA. Reconstituted extract (0.5 μL) was mixed with 0.5 μL of matrix (10 mg/mL alpha-cyano-4-hydroxy-trans-cinnamic acid in 50% ACN/0.1% TFA), spotted on a stainless steel MS plate, and air dried. Samples were analyzed using a Voyager DE-Pro MALDI-TOF mass spectrometer (Applied Biosystems) operated in the delayed extraction/reflector mode with an accelerating voltage of 20 kV, grid voltage setting of 72%, and a 50 ns delay. Five spectra (50 to 100 laser shots/spectrum) were obtained for each sample. Trypsin peptides T4 and T7 were used for internal calibration. External calibration was performed using a Sequazyme Peptide Mass Standard kit (Applied Biosystems. Peptide mass fingerprinting was conducted with the database search tool MS-Fit in the program Protein Prospector. Beside peptide mass fingerprinting the identification of novel Enoyl CoA isomerase was carried out by sequencing the tryptic peptides on a hybrid quadrupole time-of-flight mass spectrometer QSTAR (AB/MDS-SCIEX) fitted with nanospray source. Tryptic peptide mixture was desalted on pre-column packed with C18 beads and then separated via a linear gradient of increasing acetonitrile at a flow rate of ˜200 nl/min directly injected into the source. MS scans were collected in automatic mode followed by MS/MS scans of the two highest intensity peptides. All MS/MS spectra identifying proteins or peptides reported were the most probable candidates in a non-redundant FASTA database and were manually inspected for accuracy.

### Example 11

### Western Blot Analysis of proteins from Human and Swine Model

Western Blot analysis was carried out in accordance with procedures described by Van Eyk et al. (Circ. Res. 1998 82:261-271). Briefly, whole tissue homogenates were used for Western Blot analysis of 1D or 2D SDS-PAGE gels. 5 μg of total protein was used for SDS-PAGE (12.5%) and 20 μg of total protein was used for 2-DE gels (pH 3-10, 12.5%). In order to ensure reproducibility of results, Western Blot analysis of 2-DE of ventricular tissue was conducted on human and swine samples (human controls (n=5);ICM (n=4); swine 6 week SHAM (n=5), 6 week LAD (n=5)) distinct samples. Immunodetection was carried out using antibodies against:
TnI: 8I-7 (Spectral Diagnostics), Peptide 1 (BiosPacific);
Desmin: DE-U-10(Sigma);
TnC: 1A2(Biodesign);
MLC1: 1-LC14(Spectral Diagnostics);
TnT: Peptide 3 and Peptide 1(BiosPacific)

1. A protein profile indicative of heart failure in a subject comprising an altered state of one or more proteins selected from of any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3 and T-complex protein 1 theta subunit.

2. A protein profile indicative of heart failure in a subject comprising altered states of two or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.

3. The protein profile of claim 2 comprising altered states of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.

4. A method for diagnosing heart failure in a subject comprising measuring in a biological sample of the subject the protein profile of any of claims 1 through 3 and comparing the measured protein profile to a profile of the same proteins in a healthy control wherein an elevation in protein levels of any of 14-3-3 protein gamma, beta lactoglobulin 1A and 1C, 2-oxoisovalerate dehydrogenase beta, chloride intracellular channel protein 1, cytochrome b5, F-actin capping protein beta 1, glycogen phosphorylase, HSP 27, hUNC-45, MTCBP-1, long chain fatty acid CoA ligase 1, 6-phosphofructokinase, NADH-ubiquinone oxidoreductase 30 kDa subunit, NADH-ubiquinone oxidoreductase 75 kDa subunit, peroxiredoxin 4, proliferating cell nuclear antigen, protein disulphide isomerase, TnI and/or TnC and/or a decrease in protein levels of any of alpha-actinin, alpha-enolase, annexin V, aspartate aminotransferase, ATP synthase alpha chain, cytochrome C oxidase VA, desmin, dihydrolipoamide dehydrogenase, elastase IIIB, fructose-bisphosphate aldolase A, fumarate hydratase, GRP78, moesin, ventricular MLCl, MLC2, NADH ubiquinone oxidoreductase 51 kDa subunit, stathmin 3, T-complex protein 1 theta subunit, tropomyosin alpha 1, tropomyosin beta, troponin T, vimentin and/or the degraded form of MRP 1 and/or a shift of tubulin alpha, tubulin beta towards the myofilament fraction of the myocytes and/or a shift of novel enoyl CoA isomerase to its acidic form and/or a shift of the ratio between metavinculin and vinculin towards metavinculin and/or shift of HSP 90 alpha to its acidic form, as compared to the protein profile of the healthy control is indicative of heart failure.

5. A method for monitoring a subject with heart failure or at risk for heart failure comprising:
(a) measuring in the subject at a first selected time point the protein profile of any of claims 1 through 3;
(b) measuring in the subject at a second selected time point subsequent to said first selected time point the same protein profile of step (a); and
(c) comparing the measured protein profile of step (a) and step (b) to assess for changes in the profile between the first selected time and the second selected time in the subject.

6. The method of claim 5 further comprising administering to the subject a treatment for heart failure between step (a) and step (b) so that efficacy of the treatment as determined by a change in the profiles of step (a) and step (b) in the subject can be ascertained.

7. A method for treating a subject with heart failure comprising administering to the subject an agent which modulates the state of a protein of the protein profile of any of claims 1 through 3.

8. A method for staging progression of heart failure in a subject suffering from heart failure comprising measuring in a biological sample of the subject the protein profile of any of claims 1 through 3 and comparing the measured protein profile to a profile of the same proteins in diseased controls from various stages of heart failure so that the stage of progression of heart failure of the subject can be determined.

9. A method for screening for an agent which modulates heart failure comprising assessing an agent's ability to modulate a state of one or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.

10. A method for identifying agents that modulate progression of heart failure comprising:
(a) administering an agent to a LAD-ligated swine;
(b) measuring a protein profile of any of claim 1 through 3 in a biological sample obtained from the LAD-ligated swine; and
(c) comparing the measured protein profile of step (b) with a corresponding protein profile of a control, wherein a change in the measured protein profile of step (b) compared to the corresponding protein profile in the control is indicative of the agent being a modulator of heart failure.

11. A protein profile indicative of a selected stage of heart failure in a subject comprising an altered state of one or more proteins selected from of any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3 and T-complex protein 1 theta subunit.

12. A protein profile indicative of a selected stage heart failure in a subject comprising altered states of two or more proteins selected from any of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acid CoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.

13. The protein profile of claim 12 comprising altered states of 6-phosphofructokinase, 14-3-3 protein gamma, alpha-enolase, beta-lactoglobulin 1A and 1C, chloride intracellular channel protein 1, cytochrome b5, dihydrolipoamide dehydrogenase, elastase IIIB, F-actin capping protein beta 1, fructose biphosphate aldolase, fumarate hydratase, 78 kDA glucose-related protein (GRP 78), heat shock protein HSP 90 alpha (HSP 90), human striated muscle UNC 45 (hUNC45), moesin, MTCBP-1, conjugate export pump protein (MRP 1), ventricular myosin light chain 1, NADH ubiquinone oxidoreductase 30 kDA subunit, NADH ubiquinone oxidoreductase 51 kDA subunit, NADH ubiquinone oxidoreductase 75 kDa subunit, novel enoyl CoA isomerase, 2-oxoisovalerate dehydrogenase beta, protein disulfide isomerase, peroxiredoxin 4, stathmin 3, T-complex protein 1 theta subunit, alpha-actinin, annexin V, aspartate aminotransferase, ATP synthase alpha chain, cytochrome C oxidase VA, desmin, glycogen phosphorylase, heat shock protein 27 (HSP27), long chain fatty acidCoA ligase 1, myosin light chain 2, proliferating cell nuclear antigen, troponin T (TnT), troponin I (TnI), troponin C (TnC), tropomyosin alpha 1, tropomyosin beta, tubulin alpha, tubulin beta, vimentin, and (meta)vinculin.
